(19) United States
(12) Patent Application Publication
(10) Pub. No.: US 2007/0178554 A1
(43) Pub. Date: Aug. 2, 2007

(54) Orthogonal Aminoacyl Synthetase-tRNA Pairs for Incorporating Unnatural Amino Acids Into Proteins

(76) Inventors: Nima Shiva, Los Angeles, CA (US); Mark W. Nowak, Pasadena, CA (US); Niki Zacharias, Monrovia, CA (US)

Correspondence Address: SHELDON MAK ROSE & ANDERSON PC, 100 East Corson Street, Third Floor, PASADENA, CA, 91103-3842, US

(21) Appl. No.: 11/670,354

(22) Filed: Feb. 1, 2007

Related U.S. Application Data
(60) Provisional application No. 60/764,431, filed on Feb. 1, 2006.

(51) Int. Cl. C12P 21/06; C07K 14/47; C12N 15/74

(52) U.S. Cl. 435/69.1; 435/320.1; 435/455; 435/325; 530/350

(57) ABSTRACT

Methods of incorporating unnatural amino acids into proteins in eukaryotic cells using an orthogonal aminoacyl synthetase and an orthogonal tRNA derived from Lactococcus lactis.

### BACKGROUND

Proteins in virtually all organisms, and in all higher organisms, are made from twenty amino acids. In vitro studies are often performed to study the effects on protein structure and function of changes to particular amino acids in a protein, since such experiments can often be performed more reproducibly than in vivo studies. The effects of changes to particular amino acids in a protein in vivo, however, cannot always be predicted from in vitro studies. The structure and function of proteins in vivo is therefore preferably studied in living cells.

The ability to probe the function or effect of a particular amino acid in a protein in vivo has generally been limited to substituting one of the remaining 19 natural amino acids for an amino acid of interest. In recent years, however, unnatural amino acids have been incorporated into proteins in order to gain a better understanding of protein structure and function. Lei Wang and Peter Schultz have reported that unnatural amino acids can be incorporated into Escherichia coli using an aminoacyl synthetase derived from Methanococcus jannaschii, for example [P. G. Schultz and L. Wang, Expanding the Genetic Code, Angew. Chem. Int. Ed., 44, 34-66 (2005)].

The orthogonality of an aminoacyl synthetase or tRNA molecule from one species cannot be predicted a priori, however. In vivo β-lactamase complementation assays showed that the amber suppressor tRNATyrCUA derived from both S. cerevisiae and humans is not orthogonal in E. coli [see, e.g., L. Wang, T. J. Magliery, D. R. Liu and P. G. Schultz, J. Am. Chem. Soc., 122:5010 (2000)]. There remains a need, therefore, for additional systems for incorporating unnatural amino acids into proteins in cells, in particular in eukaryotic cells.

### SUMMARY

The present invention includes systems, methods, and compositions for the site-specific incorporation of unnatural amino acids directly into proteins both in vivo and in vitro. The compositions of the present invention include orthogonal aminoacyl-tRNA synthetases (O-RS molecules) derived from L. lactis which preferentially aminoacylate orthogonal tRNA molecules (O-tRNAs) with an unnatural amino acid in a eukaryotic translation system. In one aspect, the present invention comprises a translation system for incorporating unnatural amino acids into proteins. The present translation system comprises translation components, such as ribosomes, aminoacyl synthetases, and tRNAs, derived from a eukaryotic organism and an aminoacyl synthetase/tRNA pair derived from Lactococcus lactis, Gluconobacter oxydans or Rhodospirullum rubrum. The aminoacyl synthetase and tRNA of the aminoacyl synthetase/tRNA pair are orthogonal with respect to the translation components of the system, and this tRNA can be aminoacylated with an unnatural amino acid by the aminoacyl synthetase with enhanced efficiency as compared to aminoacylation of the tRNA with a natural amino acid. The tRNA comprises an anticodon loop having a sequence that specifically binds to a selector codon, which can be for example an amber codon, an opal codon, an ocher codon, or a four base codon. The aminoacyl synthetase/tRNA pair is preferably derived from Lactococcus lactis, and the aminoacyl synthetase is preferably derived from a tyrosyl aminoacyl synthetase.

The unnatural amino acid that's incorporated into a protein according to the present methods can be for example, a tyrosine analog, a glutamine analog, a phenylalanine analog, serine analog, a threonine analog, a β-amino acid, or a cyclic amino acid other than proline. hydroxy methionine, norvaline, O-methylserine. crotylglycine, hydroxy leucine, allo-isoleucine, norleucine, α-aminobutyric acid, t-butylalanine, hydroxy glycine, hydroxy serine, F-alanine, hydroxy tyrosine, homotyrosine, 2-F-tyrosine, 3-F-tyrosine, 4-methyl-phenylalanine, 4-methoxy-phenylalanine, 3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, cyclohexanol-alanine, hydroxyl alanine, hydroxy phenylalanine, hydroxy valine, hydroxy isoleucine hydroxyl glutamine, thienylalanine, pyrrole alanine, N_{T}-methyl-histidine, 2-amino-5-oxohexanoic acid, norvaline, norleucine, 3,5-F₂-phenyalanine, cyclohexyalanine, 4-Cl-phenyalanine, p-azido-phenylalanine, o-azido-phenylalanine, O-4-allyl-L-tyrosine, 2-amino-4-pentanoic acid, and 2-amino-5-oxohexanoic acid.

Alternatively, the unnatural amino acid can be a derivative of a natural amino acid comprising a substitution or addition selected from the group consisting of an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, oc-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, and an amino group. The unnatural amino acid can also be a derivative of a natural amino acid comprising an addition selected from the group consisting of a photoactivatable cross-linker, a spin-label, a fluorescent label, a radioactive label, biotin, a biotin analog, and a photocleavable group.

In one embodiment, the translation components of the present system comprise the endogenous translation components of a cell, and the aminoacyl synthetase/tRNA pair is present in the cell. The cell can be, for example, a yeast cell, an insect cell, or a mammalian cell, such as a CHO or human cell. In this embodiment, the aminoacyl synthetase/tRNA pair can be produced by introducing one or more nucleic acid molecules into the cell that comprise sequences that encode the aminoacyl synthetase and the tRNA, such as the sequences set forth as SEQ ID NOS. 1-11 herein.

In this embodiment, the present invention can comprise a method of incorporating an unnatural amino acid into a protein in a eukaryotic cell, comprising the steps of providing a eukaryotic cell having an aminoacyl synthetase/tRNA pair as described above; providing an unnatural amino acid; and producing the protein having the unnatural amino acid incorporated therein. In a preferred embodiment, the aminoacyl synthetase/tRNA pair can be provided by transfecting both a nucleic acid molecule that encodes an aminoacyl synthetase derived from Lactococcus lactis and a nucleic acid molecule that encodes a tRNA derived from Lactococcus lactis into the cell.

The present invention can further comprise a vector for use in this method, the vector a first nucleic acid molecule comprising a first nucleic acid sequence that encodes an aminoacyl synthetase derived from Lactococcus lactis; and a second nucleic acid molecule comprising a second nucleic acid sequence that encodes a tRNA derived from Lactococcus lactis that is aminoacylated with an unnatural amino acid by the aminoacyl synthetase derived from Lactococcus lactis, wherein the tRNA comprises an anticodon loop having a sequence that specifically binds a selector codon of an mRNA molecule. These nucleic acid molecules can be present in the same or different plasmids, for example.

### DRAWINGS

These and other features, aspects and advantages of the present invention will become better understood with regard to the following description, appended claims, and accompanying figures where:

FIGS. 1A-1D illustrate the incorporation of an unnatural amino acid into a protein using an O-RS/O-tRNA pair.

FIG. 2 is a bar chart showing bovine TyrRS aminoacylation of human and bacterial tyrosyl tRNAs.

FIG. 3 is a bar chart showing aminoacylation of human and bacterial tRNA by several bacterial synthetases.

FIG. 4A shows an electrophysiological measurement of a CHO cell transfected with a plasmid encoding hERG WT. The X-axis shows a time period of 2 seconds and the Y-axis shows a current level of 500 nA.

FIG. 4B shows an electrophysiological measurement of a CHO cell transfected with a plasmid encoding hERG 652TAG mutant as well as with plasmids encoding L. lactis aminoacyl synthetase and L. lactis tRNA_{CUA}. The X-axis shows a time period of 2 seconds and the Y-axis shows a current level of 500 nA.

FIG. 4C shows an electrophysiological measurement of a CHO cell transfected with a plasmid encoding hERG 652TAG mutant and with a plasmid encoding L. lactis tRNA_{CUA} in the absence of L. lactis aminoacyl synthetase. The X-axis shows a time period of 2 seconds and the Y-axis shows a current level of 500 nA.

FIG. 5 illustrates a strategy for generating a library of L. lactis aminoacyl synthetase mutants.

FIG. 6A depicts plasmid ptRNA_{CUA}/ADH1-TyrRS.

FIG. 6B depicts plasmid pYeastSelection (GAL4).

All dimensions specified in this disclosure are by way of example only and are not intended to be limiting. Further, the proportions shown in these Figures are not necessarily to scale.

### DESCRIPTION

The present systems and methods enable control over the incorporation of unnatural amino acids into proteins expressed in eukaryotic cells, in particular in mammalian cells, in a site-directed manner. The compositions used in the present systems and methods comprise translation components that expand the number of genetically encoded amino acids in such eukaryotic cells. Such components include, inter alia, , aminoacyl synthetases and tRNA derived from L. lactis as well as unnatural amino acids. Aminoacyl synthetases and tRNA molecules derived from L. lactis are orthogonal with respect to the translation components of eukaryotic cells, and such aminoacyl synthetases conjugate an unnatural amino acid to a tRNA derived from L. lactis to create an aminoacylated tRNA that recognizes a selector codon, such as an amber stop codon, placed in frame at any position in an mRNA molecule coding for a protein of interest.

Our data indicate that the RS/tRNA pairs from L. lactis, G. oxydans and R. rubrum, in particular TyrRS/tRNA pairs, are orthogonal to eukaryotic RS/tRNA pairs. The fact that L. acidophilus and L. casei TyrRS/tRNA pairs were not found to be orthogonal to a mammalian translation system indicates that not all bacterial TyrRS/tRNA pairs are orthogonal to mammalian TyrRS/tRNA pairs, and that it is not obvious that a bacterial RS/tRNA pair (i.e. for a particular amino acid) will necessarily be orthogonal to the corresponding RS/tRNA eukaryotic pair. This was also shown by Shiba et al., who found that E. coli and human Gly RS/tRNA pairs are orthogonal but that Ala RS/tRNA pairs are not [Shiba, K, et al., Human glycyl-tRNA synthetase: Wide divergence of primary structure from bacterial counterpart and species-specific aminoacylation, J Biol Chem 269:30049-55 (1994)].

Definitions

As used herein, the following terms and variations thereof have the meanings given below, unless a different meaning is clearly intended by the context in which such term is used.

“About” and “approximately” shall generally mean an acceptable degree of error for the quantity measured given the nature or precision of the measurements. Typical, exemplary degrees of error are within 20 percent (%), preferably within 10%, and more preferably within 5% of a given value or range of values. Alternatively, and particularly in biological systems, the terms “about” and “approximately” can mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

“Analog” means a molecule which resembles another molecule in structure, such as a molecule which comprises a portion of the chemical structure or polymer sequence of another molecule, but which is not identical to or an isomer of such other molecule.

“Derived from” and “derivative” refer to a composition or component which is: (1) isolated from a source, such as from a particular organism; (2) isolated from a source and then modified; or (3) formed from a particular molecule or starting material, i.e. a modified form of such starting molecule or material. Also included are compositions and components that are generated (e.g., chemically synthesized or recombinantly produced) using sequence, chemical composition, structure, or other information about such a derived composition or component.

“Expression system” means a host cell and compatible vector, e.g. for the expression of a protein coded for by foreign DNA carried by the vector and introduced to the host cell.

“Eukaryote” and “eukaryotic” refer to organisms belonging to the phylogenetic domain Eucarya, including those belonging to the taxonomic kingdoms Animalia and Fungi, such as animals (e.g., mammals, insects, reptiles, and birds) and fungi (such as yeasts). Particularly preferred cells for use in the present method include those of eukaryotes from the taxonomic classes Mammalia and Amphibia, such as human cells, CHO cells, and Xenopus oocytes.

“Identical” or percent “identity,” in the context of two or more nucleic acid or polypeptide sequences, refer to two or more sequences that are the same or have a specified percentage of amino acid residues or nucleotides that are the same, when compared and aligned for maximum correspondence, as measured using one of the sequence comparison algorithms known to persons of skill in the art. “Substantially identical,” in the context of two nucleic acids or polypeptides (e.g., DNAs encoding an O-tRNA or O-RS, or the amino acid sequence of an O-RS) refers to two or more nucleic acid or amino acid sequences that have at least about 60%, preferably 80%, most preferably 90-95% nucleotide or amino acid residue identity, when compared and aligned for maximum correspondence, as measured using a sequence comparison algorithm. Preferably, “substantial identity” exists over a region of the sequences that is at least about 50 residues in length, more preferably over a region of at least about 100 residues, and most preferably the sequences are substantially identical over at least about 150 residues, or over the full length of the two sequences to be compared.

“Natural amino acid” means selenocysteine and the following twenty alpha-amino acids: alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

“Negative selection marker” refers to a detectable indicator than, when present, e.g., expressed in a cell, activated or the like, allows identification of an organism that does not possess a particular property (e.g., as compared to an organism which does possess the property). A “positive selection marker” conversely refers to an indicator than when present, e.g., expressed, activated or the like, results in identification of an organism with the positive selection marker from those without the positive selection marker.

“Orthogonal” refers either to a tRNA molecule or to an aminoacyl synthetase molecule which reacts with reduced efficiency with the endogenous components of a translation system, either in vivo or in vitro. Reduced efficiency refers to a lesser ability of an orthogonal component to aminoacylate or be aminoacylated by an endogenous component of a cell or other translation system, and can be, e.g., to a level of less than 20% as efficient as an endogoenous component, less than 10% as efficient, less than 5% as efficient, or less than 1% as efficient, with efficiency being measured by K_{cat}/Kₘ. For example, an orthogonal tRNA in a translation system of interest is aminoacylated by any endogenous aminoacyl synthetase of the translation system with reduced or even zero efficiency, when compared to aminoacylation of an endogenous tRNA by the endogenous aminoacyl synthetase of the translation system. In another example, an orthogonal aminoacyl synthetase aminoacylates any endogenous tRNA in the translation system of interest with reduced or even zero efficiency as compared to aminoacylation of the endogenous tRNA by an endogenous aminoacyl synthetase.

“O-RS” means orthogonal aminoacyl-tRNA synthetase. “RS” means an aminoacyl-tRNA synthetase (i.e., aminoacyl synthetase).

“O-tRNA” means orthogonal tRNA.

“Preferential aminoacylation” means aminoacylation of a tRNA molecule with greater efficiency, i.e. with a higher K_{cat}/Kₘ. Preferential aminoacylation is preferably at an efficiency of greater than about 70% efficient, and more preferably of greater than about 80% efficient. In preferred embodiments, preferential aminoacylation occurs at an efficiency of greater than about 90%, such as at an efficiency of about 95%-99% or higher. With respect to an O-RS, preferential aminoacylation generally refers to the aminoacylation of O-tRNA with an unnatural amino acid at greater efficiency compared to aminoacylation of a naturally occurring tRNA with the amino acid.

“Reporter” means a measurable composition or characteristic of a composition, or another component of a system which codes for or results in the production of such a composition. For example, a reporter can include a green fluorescent protein, firefly luciferase protein, β-galactosidase or alcohol dehydrogenase, or can be a nucleic acid which encodes such a protein.

“Selector codon” means a codon (i.e., a series of 3 or more nucleic acids) recognized by an O-tRNA in the translation process and not recognized by an endogenous tRNA. The O-tRNA anticodon loop recognizes the selector codon on an mRNA so that the amino acid it carries, e.g. an unnatural amino acid, is incorporated at the site in the polypeptide encoded by the selector codon.

A “suppressor tRNA” is a tRNA that alters the reading of a messenger RNA (mRNA) in a given translation system, in particular by recognizing a stop codon or other nonsense codon and supplying an amino acid, thereby allowing the transcription of codons located 3′ of the stop or nonsense codon.

“Translation system” refers to the biochemical components, e.g. of a cell, necessary to incorporate an amino acid into a growing polypeptide chain (protein). Such components include, e.g., ribosomes, tRNAs, synthetases, and mRNA. The components of a translation system can be present either in vivo or in vitro.

“Unnatural amino acid” means any amino acid, amino acid derivative, amino acid analog, α-hydroxy acid, or other molecule other than a natural amino acid which can be incorporated into a polypeptide chain with an O-tRNA/O-RS pair and which allows extension of the polypeptide chain.

As used herein, the term “comprise” and variations of the term, such as “comprising” and “comprises,” are not intended to exclude other additives, components, integers or steps. The terms “a,” “an,” and “the” and similar referents used herein are to be construed to cover both the singular and the plural unless their usage in context indicates otherwise.

Orthogonal tRNAs and Orthogonal Aminoacyl-tRNA Synthetases

An orthogonal tRNA for use in the present systems and methods recognizes a selector codon and is preferentially aminoacylated in a translation system with an unnatural amino acid by an orthogonal aminoacyl-tRNA synthetase. In one embodiment, the O-tRNA comprises a nucleic acid which is encoded by a polynucleotide sequence selected from the group consisting of SEQ ID NOS. 8-11 and/or a complementary polynucleotide sequence thereto. An O-tRNA can be, e.g., a suppressor tRNA, a frameshift tRNA. Mutations can be introduced into O-tRNAs at a specific position or positions, e.g., at one or more nonconservative positions, conservative positions, randomized positions, or a combination of such positions in a desired loop of a tRNA, e.g., in an anticodon loop, D arm, Variable loop, T arm, acceptor stem, or in a combination of loops or regions, or in all the loops.

In order to specifically incorporate an unnatural amino acid into a protein in vivo, the substrate specificity of an aminoacyl-tRNA synthetase is altered so that only the desired unnatural amino acid, but not any of the common 20 amino acids, are charged to the corresponding O-tRNA. If the orthogonal synthetase is promiscuous, it will result in mutant proteins with a mixture of natural and unnatural amino acids at the target position. The efficiency of incorporation of an unnatural amino acid into a protein with the present systems and methods can be, e.g., greater than about 75%, greater than about 85%, greater than about 95%, or greater than about 99%. Preferably, orthogonal aminoacyl-tRNA synthetases have improved or enhanced enzymatic properties, e.g., the Kₘ is lower, the k_{cat} is higher, and/or the value of k_{cat}/Kₘ is higher, for the unnatural amino acid as compared to a naturally occurring amino acid, e.g., one of the 20 known amino acids.

An orthogonal pair is composed of an O-tRNA and an O-RS. The O-tRNA is not preferentially acylated by endogenous synthetases, and is capable of decoding a selector codon, as described above. The O-RS of an O-RS/O-tRNA pair recognizes the O-tRNA and preferentially aminoacylates the O-tRNA with an unnatural amino acid. The development of multiple orthogonal tRNA/synthetase pairs can allow the simultaneous incorporation of multiple unnatural amino acids using different codons into the same polypeptide/protein.

Sequences of O-tRNA and O-RS Molecules

The O-tRNAs, O-RS molecules, and other components of the present methods and systems comprise amino acid sequences and corresponding nucleic acid sequences. One of skill in the art will appreciate that the invention is not limited to those specific sequences disclosed herein, and that many variants of such disclosed sequences are possible. For example, conservative variations of the disclosed sequences that yield a component of similar or identical functionally can be utilized in the present systems. The addition of sequences which do not alter the encoded activity of a nucleic acid molecule, such as the addition of a non-functional sequence, is a conservative variation of the basic nucleic acid.

Owing to the degeneracy of the genetic code, “silent substitutions” (i.e., substitutions in a nucleic acid sequence which do not result in an alteration in an encoded polypeptide) are an implied feature of every nucleic acid sequence which encodes an amino acid. Similarly, “conservative amino acid substitutions,” in one or a few amino acids in an amino acid sequence can be readily identified as being equivalent to a disclosed construct.

Conservative substitutions are exemplified in Table 1 below. One of skill will recognize that individual substitutions, deletions or additions which alter, add, or delete a single amino acid or a small percentage of amino acids (typically less than 5%, more typically less than 4%, 2%, or 1%) in an encoded sequence are “conservative variations” where the alterations result in the deletion of an amino acid, addition of an amino acid, or substitution of an amino acid with a chemically similar amino acid. Thus, conservative variations of a polypeptide sequence of the present invention include substitutions of a small percentage, typically less than 5%, more typically less than 2% or 1%, of the amino acids of the polypeptide sequence, preferably with an amino acid of the same conservative substitution group.

TABLE 1
Conservative Substitution Groups

| | | | | |
|---|---|---|---|---|
| 1 | Alanine (A) | Serine (S) | Threonine (T) | |
| 2 | Aspartic acid (D) | Glutamic acid (E) | | |
| 3 | Asparagine (N) | Glutamine (O) | | |
| 4 | Arginine (R) | Lysine (K) | | |
| 5 | Isoleucine (I) | Leucine (L) | Methionine (M) | Valine (V) |
| 6 | Pheaylalanine (F) | Tyrosine (Y) | Trytophan (W) | |

Variants of the present polynucleotide sequences, where the variants hybridize to at least one disclosed sequence, can likewise be used. Comparative hybridization can be used to identify such nucleic acids of the invention, and this comparative hybridization method is a preferred method of distinguishing nucleic acids of the invention. In addition, target nucleic acids which hybridize to the nucleic acids represented by SEQ ID NOS. 1-11 under high, ultra-high and ultra-ultra high stringency conditions are a feature of the invention. Examples of such nucleic acids include those with one or a few silent or conservative nucleic acid substitutions as compared to a given nucleic acid sequence.

A test nucleic acid is said to specifically hybridize to a probe nucleic acid when it hybridizes at least ½ as well to the probe as to the perfectly matched complementary target, i.e., with a signal to noise ratio at least ½ as high as hybridization of the probe to the target under conditions in which the perfectly matched probe binds to the perfectly matched complementary target with a signal to noise ratio that is at least about 5 times to 10 times as high as that observed for hybridization to any of the unmatched target nucleic acids.

Nucleic acids “hybridize” when they associate, typically in solution. Nucleic acids hybridize due to a variety of well characterized physico-chemical forces, such as hydrogen bonding, solvent exclusion, and base stacking. An extensive guide to the hybridization of nucleic acids is found in Tijssen, Laboratory Techniques in Biochemistry and Molecular Biology—Hybridization with Nucleic Acid Probes, part I, chapter 2, “Overview of principles of hybridization and the strategy of nucleic acid probe assays,” Elsevier, New York, (1993), as well as in Ausubel, infra. Hames and Higgins, “Gene Probes” and “Gene Probes 2,” 1 IRL Press at Oxford University Press, Oxford, England, provide details on the synthesis, labeling, detection and quantification of DNA and RNA, including oligonucleotides.

An example of stringent hybridization conditions for hybridization of complementary nucleic acids which have more than 100 complementary residues on a filter in a Southern or Northern blot is 50% formalin with 1 mg of heparin at 42° C., with the hybridization being carried out overnight. An example of stringent wash conditions is a 0.2×SSC wash at 65° C. for 15 minutes (see, Sambrook, infra for a description of SSC buffer). Often the high stringency wash is preceded by a low stringency wash to remove background probe signal. An example low stringency wash is 2×SSC at 40° C. for 15 minutes. In general, a signal to noise ratio of 5 times (or higher) than that observed for an unrelated probe in the particular hybridization assay indicates detection of a specific hybridization.

“Stringent hybridization wash conditions” in the context of nucleic acid hybridization experiments such as Southern and northern hybridizations are sequence dependent, and are different under different environmental parameters. Stringent hybridization and wash conditions can easily be determined empirically for any test nucleic acid. For example, in determining highly stringent hybridization and wash conditions, the hybridization and wash conditions are gradually increased (e.g., by increasing temperature, decreasing salt concentration, increasing detergent concentration and/or increasing the concentration of organic solvents such as formalin in the hybridization or wash), until a selected set of criteria are met. For example, the hybridization and wash conditions are gradually increased until a probe binds to a perfectly matched complementary target with a signal to noise ratio that is at least 5 times as high as that observed for hybridization of the probe to an unmatched target.

“Very stringent” conditions are selected to be equal to the thermal melting point (Tₘ) for a particular probe. The Tₘ is the temperature (under defined ionic strength and pH) at which 50% of the test sequence hybridizes to a perfectly matched probe. For the purposes of the present invention, generally, “highly stringent” hybridization and wash conditions are selected to be about 5° C. lower than the Tm for the specific sequence at a defined ionic strength and pH.

“Ultra high-stringency” hybridization and wash conditions are those in which the stringency of hybridization and wash conditions are increased until the signal to noise ratio for binding of the probe to the perfectly matched complementary target nucleic acid is at least 10 times as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-high stringency conditions.

Similarly, even higher levels of stringency can be determined by gradually increasing the hybridization and/or wash conditions of the relevant hybridization assay. For example, those in which the stringency of hybridization and wash conditions are increased until the signal to noise ratio for binding of the probe to the perfectly matched complementary target nucleic acid is at least 10 times, 20 times, 50 times, 100 times, or 500 times or more as high as that observed for hybridization to any of the unmatched target nucleic acids. A target nucleic acid which hybridizes to a probe under such conditions, with a signal to noise ratio of at least ½ that of the perfectly matched complementary target nucleic acid is said to bind to the probe under ultra-ultra-high stringency conditions.

Nucleic acids which do not hybridize to each other under stringent conditions are still substantially identical if the polypeptides which they encode are substantially identical. This occurs, e.g., when a copy of a nucleic acid is created using the maximum codon degeneracy permitted by the genetic code.

Selector Codons

Selector codons in mRNA molecules allow unnatural amino acids to be incorporated into proteins using O-RS/O-tRNA pairs. The 64 genetic codons code for 20 amino acids and 3 stop codons. Because only one stop codon is needed for translational termination, the other two can in principle be used to encode nonproteinogenic amino acids. The amber stop codon, UAG, has been successfully used in in vitro biosynthetic system and in Xenopus oocytes to direct the incorporation of unnatural amino acids. Different species preferentially use different codons for their natural amino acids, and such preferentiality is optionally utilized in designing/choosing the selector codons herein. For example, a selector codon includes, e.g., a unique three base codon, a nonsense codon, such as a stop codon, e.g., an amber codon, or an opal codon, an unnatural codon, a four (or more) base codon or the like. A number of selector codons can be introduced into a desired nucleic acid sequence, e.g., one or more, two or more, or more than three. As a result, a number of unnatural amino acids (the same and/or different unnatural amino acids) can be incorporated precisely into a polypeptide chain.

Selector codons preferably allow the presence or functionality of an O-RS/O-tRNA pair to be detected and/or studied. Selector codons can be, for example, nonsense codons such as stop codons, e.g., amber (TAG/UAG), ochre (TAA/UAA), and opal (TGA/UGA) codons, in which case the presence and functionality of an O-RS/O-tRNA pair can be detected through the detection of a full length protein coded for by an mRNA comprising the selector codon. Other selector codons include codons with four or more bases. For a given system a selector codon can also include one of the natural three base codons, if the system does not use the natural three base codon, i.e. a system lacking a tRNA that recognizes the natural three base codon.

Although discussed with reference to unnatural amino acids herein, it will be appreciated that a similar strategy can be used to incorporate a natural amino acid in response to a particular selector codon. That is, a synthetase can be modified to load a natural amino acid onto an orthogonal tRNA that recognizes a selector codon in a manner similar to the loading of an unnatural amino acid as described herein.

In one embodiment, the present methods involve the use of a selector codon that is a stop codon for the incorporation of unnatural amino acids in vivo. For example, an O-tRNA is generated that recognizes the stop codon, sucha as UAG in E. coli, and is aminoacylated by an O-RS with a desired unnatural amino acid. This O-tRNA is not recognized by the naturally occurring aminoacyl-tRNA synthetases of the host cell. Conventional site-directed mutagenesis can be used to introduce the stop codon, e.g., TAG, at the site of interest in the nucleic acid sequence [see, e.g., Sayers, J. R., Schmidt, W. Eckstein, F., 5′, 3′ Exonuclease in phosphorothioate-based oligonucleotide-directed mutagenesis, Nucleic Acids Res, 791-802 (1988)]. When the O-RS, O-tRNA and the mutant nucleic acid sequence are combined in vivo, the unnatural amino acid is incorporated in response to, e.g., the UAG codon to give a protein containing the unnatural amino acid at the specified position.

Selector codons can also comprise four or more base codons, such as, four, five, six or more. Examples of four base codons include, e.g., AGGA, CUAG, UAGA, CCCU and the like. Examples of five base codons include, e.g., AGGAC, CCCCU, CCCUC, CUAGA, CUACU, UAGGC and the like. For example, in the presence of O-tRNAs comprising a special frameshift suppressor tRNA, e.g., anticodon loops with 8-10 nucleotides, a four or more base codon is read as a codon for a single amino acid. In other embodiments, anticodon loops of O-tRNAs can decode, e.g., at least a four-base codon, at least a five-base codon, or at least a six-base codon or more. Since there are 256 possible four-base codons, multiple unnatural amino acids can be encoded in the same cell using codons comprising four or more bases [see, J. Christopher Anderson et al., Exploring the Limits of Codon and Anticodon Size, Chemistry and Biology, Vol. 9, 237-244 (2002); Thomas J. Magliery, Expanding the Genetic Code: Selection of Efficient Suppressors of Four-base Codons and Identification of “Shifty” Four-base Codons with a Library Approach in E. coli, J. Mol. Biol. 307: 755-769 (2001)].

The present methods can also include using extended codons based on frameshift suppression. Four or more base codons can insert, e.g., one or multiple unnatural amino acids into the same protein. For example, four-base codons have been used to incorporate unnatural amino acids into proteins using in vitro biosynthetic methods [see, e.g., C. H. Ma, W. Kudlicki, O. W. Odom, G. Kramer and B. Hardesty, Biochemistry, 32:7939 (1993); T. Hohsaka, D., et al., Am. Chem. Soc., 121:34 (1999)]. The codons CGGG and AGGU were used to simultaneously incorporate 2-naphthylalanine and an NBD derivative of lysine into streptavidin in vitro with two chemically acylated frameshift suppressor tRNAs [see, e.g., T. Hohsaka, Y. Ashizuka, H. Sasaki, H. Murakami and M. Sisido, J. Am. Chem. Soc., 121:12194 (1999)]. In an in vivo study, Moore et al. examined the ability of tRNAL^{Leu} derivatives with NCUA anticodons to suppress UAGN codons (N can be U, A, G, or C), and found that the quadruplet UAGA can be decoded by a tRNA^{Leu} with a UCUA anticodon with an efficiency of 13-26% with little decoding in the 0 or −1 frame [see, B. Moore, B. C. Persson, C. C. Nelson, R. F. Gesteland and J. F. Atkins, J. Mol. Biol., 298:195 (2000)]. Extended codons based on rare codons or nonsense codons can be used to reduce missense readthrough and frameshift suppression at unwanted sites.

Unnatural amino acids can also be encoded with rare codons. For example, when the arginine concentration in an in vitro protein synthesis reaction is reduced, the rare arginine codon, AGG, has proven to be efficient for insertion of Ala by a synthetic tRNA acylated with alanine [see, e.g., C. H. Ma, W. Kudlicki, O. W. Odom, G. Kramer and B. Hardesty, Biochemistry, 32:7939 (1993)]. In this case, the synthetic tRNA competes with the naturally occurring TRNA^{Arg}, which exists as a minor species in E. coli. Some organisms do not use all triplet codons, leaving such codons available for use in the present methods when the translation system comprises translation components from such an organism. An unassigned codon AGA in Micrococcus luteus has been utilized for insertion of amino acids in an in vitro transcription/translation extract [see, e.g., A. K. Kowal and J. S. Oliver, Nucl. Acid. Res., 25:4685 (1997)]. Components of the present invention can be generated to use these rare codons in vivo.

Selector codons can also comprise unnatural nucleic acid base pairs. Unnatural base pairs incorporated into mRNA and/or tRNA molecules can expand the number of codons/anticodons available for constructing polypeptides. One extra base pair alone increases the number of triplet codons from 64 to 125. Properties of third base pairs include stable and selective base pairing, efficient enzymatic incorporation into DNA with high fidelity by a polymerase, and the efficient continued primer extension after synthesis of the nascent unnatural base pair. For in vivo usage, the unnatural nucleoside should be membrane permeable and should be phosphorylated to form the corresponding triphosphate. In addition, the increased genetic information should be stable and not destroyed by cellular enzymes.

Descriptions of unnatural base pairs which can be adapted for the present methods and systems include, e.g., that found in Hirao, et al., An unnatural base pair for incorporating amino acid analogs into protein, Nature Biotechnology, 20:177-182 (2002). Other publications are listed below. In an effort to develop an unnatural base pair satisfying all the above requirements, Schultz, Romesberg and co-workers have systematically synthesized and studied a series of unnatural hydrophobic bases. The PICS:PICS self-pair has been found to be more stable than natural base pairs, and can be efficiently incorporated into DNA by the Klenow fragment of E. coli DNA polymerase I (KF) [see, e.g., D. L. McMinn, A. K. Ogawa, Y. Q. Wu, J. Q. Liu, P. G. Schultz and F. E. Romesberg, J. Am. Chem. Soc., 121:11586 (1999); and, A. K. Ogawa, Y. Q. Wu, D. L. McMinn, J. Q. Lu, P. G. Schultz and F. E. Romesberg, J. Am. Chem. Soc., 122:3274 (2000)]. A mutant DNA polymerase has been recently evolved that can be used to replicate the PICS self pair. In addition, a 7AI self pair can be replicated using a combination of KF and pol β polymerase [see, e.g., E. J. L. Tae, Y. Q. Wu, G. Xia, P. G. Schultz and F. E. Romesberg, J. Am. Chem. Soc., 123:7439 (2001)]. A novel metallobase pair, Dipic:Py, has also been developed, which forms a stable pair upon binding Cu(II). [see, E. Meggers, P. L. Holland, W. B. Tolman, F. E. Romesberg and P. G. Schultz, J. Am. Chem. Soc., 122:10714 (2000)]. Because extended codons and unnatural codons are intrinsically orthogonal to natural codons, the methods of the present invention can take advantage of this property to generate orthogonal tRNAs for them.

Vectors

Host cells generally are genetically engineered (e.g., transformed, transduced or transfected) with vectors in order to provide O-RS and/or O-tRNA molecules in such cells and/or to produce O-RS and/or O-tRNA molecules for use in in vitro translation systems. The vector can be, for example, a cloning vector or an expression vector, and can be in the form of a plasmid, a bacterium, a virus, a naked polynucleotide, a conjugated polynucleotide, or other form. The vectors are introduced into cells and/or microorganisms by standard methods including electroporation [From et al., Proc. Natl. Acad. Sci. USA, 82:5824 (1985)], infection by viral vectors, high velocity ballistic penetration by small particles with the nucleic acid [Klein et al., Nature 327, 70-73 (1987)]. The Berger, Sambrook, and Ausubel references cited herein provide a variety of appropriate transformation methods.

Unnatural Amino Acids

A wide variety of unnatural amino acids can be used in the present compositions and methods. An unnatural amino acid can be chosen based on desired characteristics of the unnatural amino acid, for example the function of the unnatural amino acid (such as modifying protein biological properties, e.g., toxicity, biodistribution, or half life), structural properties, spectroscopic properties, chemical and/or photochemical properties, catalytic properties, or the ability to react with other molecules (either covalently or noncovalently).

An unnatural amino acid for use in the present systems and methods can be, for example, a tyrosine analog, a glutamine analog, a phenylalanine analog, serine analog, a threonine analog, a α-amino acid, or a cyclic amino acid other than proline. Unnatural amino acids can further be a derivative of a natural amino acid comprising a substitution or addition selected from the group consisting of an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, α-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, and an amino group.

In particular, the unnatural amino acid can be any of the following compounds: hydroxy methionine, norvaline, O-methylserine. crotylglycine, hydroxy leucine, allo-isoleucine, norleucine, α-aminobutyric acid, t-butylalanine, hydroxy glycine, hydroxy serine, F-alanine, hydroxy tyrosine, homotyrosine, 2-F-tyrosine, 3-F-tyrosine, 4-methyl-phenylalanine, 4-methoxy-phenylalanine, 3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, cyclohexanol-alanine, hydroxyl alanine, hydroxy phenylalanine, hydroxy valine, hydroxy isoleucine hydroxyl glutamine, thienylalanine, pyrrole alanine, N_{T}-methyl-histidine, 2-amino-5-oxohexanoic acid, norvaline, norleucine, 3,5-F₂-phenyalanine, cyclohexyalanine, 4-Cl-phenyalanine, p-azido-phenylalanine, o-azido-phenylalanine, O-4-allyl-L-tyrosine, 2-amino-4-pentanoic acid, and 2-amino-5-oxohexanoic acid.

The unnatural amino acid can also be a derivative of a natural amino acid comprising an addition selected from the group consisting of a photoactivatable cross-linker, a spin-label, a fluorescent label, a radioactive label, biotin, a biotin analog, and a photocleavable group. Further examples of unnatural amino acids can be found, for example, in the following U.S. Patent Publications, the contents of which are hereby incorporated by reference: 2003-0082575, 2005-0250183, 2003-0108885, 2005-0208536, and 2005-0009049. The synthesis of unnatural amino acids is known to those of skill in the art, and is described, e.g., in U.S. Patent Publication No. 2003-0082575.

The unnatural amino acids can, in one embodiment, comprise fluorescent moieties. Preferred compounds include those containing dansyl like dansylysine; tryptophan analogs like 7-azatryptophan; anthraniloyl containing unnatural amino acids like 3-anthraniloyl-2-amino propionic acid (AtnDap); acrylodan containing unnatural amino acids like 6-dimethylamino-2-acyl-napthalene alanine (ALADAN); coumarin containing unnatural amino acids like 2-amino-3-[6,7dimethoxy-2-oxo-2H-chromen-4-ylmethyl)-amino]-propionic acid; NBD containing unnatural amino acids like 2-amino-3-(7-nitro-benzo[1,2,5]oxadiazol-4-ylamino)propionic acid (NBD-Dap); and dipyrrometheneboron difluoride (BODIPY) containing unnatural amino acids like 2-amino-3-BODIPY-propionic acid or 2-amino-6-BODIPY-hexanoic acid. Preferred fluorescent moieties are those sensitive to the polarity of the environment to which they are exposed, i.e. fluorescent moieties whose fluorescence intensity changes depending on the polarity (hydrophilicity or hydrophobicity) of the fluorophore's environment. Such polarity-sensitive fluorophores include nitrobenzoxadiazole (NBD), acrylodan, dansyl fluorophores such as dansyl chloride, dansylalanine, and dansylysine, and some coumarin dyes.

Unnatural amino acids can, in another embodiment, be naturally occurring compounds other than the twenty-one natural alpha-amino acids found in living organisms. Because unnatural amino acids can differ from the natural amino acids only in the side chain of such molecules, some unnatural amino acids can form amide bonds with other amino acids, e.g., natural or unnatural, in the same manner in which they are formed in naturally occurring proteins. In addition to unnatural amino acids that contain novel side chains, unnatural amino acids can also comprise modified backbone structures, e.g., as illustrated by the structures of Formula II and III: 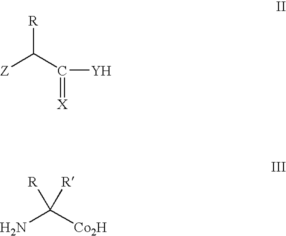
wherein Z typically comprises OH, NH₂, SH, NH—R′, or S—R′; X and Y, which can be the same or different, typically comprise S or O, and R and R′, which are optionally the same or different, typically can be any substituent other than one used in the twenty natural amino acids, as well as hydrogen. For example, unnatural amino acids can comprise substitutions in the amino or carboxyl group as illustrated by Formulas II and III. Unnatural amino acids of this type include α-hydroxy acids, α-thio acids α-aminothiocarboxylates, e.g., with side chains corresponding to the common twenty natural amino acids or unnatural side chains. In addition, substitutions at the α-carbon optionally include L, D, or α-α-disubstituted amino acids such as D-glutamate, D-alanine, D-methyl-O-tyrosine, and aminobutyric acid. Other structural alternatives include cyclic amino acids, such as proline analogs, as well as 3, 4, 6, 7, 8, and 9 membered ring proline analogs, and β and γ-amino acids such as substituted β-alanine and γ-amino butyric acid.

Unnatural amino acids can be based on natural amino acids, such as tyrosine, glutamine, and phenylalanine. Tyro sine analogs include para- substituted tyro sines, ortho-substituted tyrosines, and meta substituted tyrosines, wherein the substituted tyrosine comprises an acetyl group, a benzoyl group, an amino group, a hydrazine, an hydroxyamine, a thiol group, a carboxy group, an isopropyl group, a methyl group, a C₆-C₂₀ straight chain or branched hydrocarbon, a saturated or unsaturated hydrocarbon, an O-methyl group, a polyether group, or a nitro group. In addition, multiply substituted aryl rings are also contemplated. Glutamine analogs of the invention include, but are not limited to, α-hydroxy derivatives, γ-substituted derivatives, cyclic derivatives, and amide substituted glutamine derivatives. Example phenylalanine analogs include meta-substituted phenylalanines, wherein the substituent comprises a hydroxy group, a methoxy group, a methyl group, an allyl group, an acetyl group, or the like. Specific examples of unnatural amino acids include, but are not limited to, O-methyl-L-tyrosine, an L-3-(2-naphthyl)alanine, a 3-methyl-phenylalanine, an O-4-allyl-L-tyrosine, a 4-propyl-L-tytosine, a tri-O-acetyl-GlcNAcp-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-iodo-phenylalanine, a p-bromophenylalanine, a p-amino-L-phenylalanine, and an isopropyl-L-phenylalanine, and the like.

Also included are amino acids in which the reactive moiety is “caged,” such that the reactive group is activated or revealed only with certain treatment or processing, such as photolysis. For example, unnatural amino acids which have a halide on their side chain protected with either 2-nitrobenzyl, 3,5-dimethoxy-2-nitrobenzyl, or 3,5-dimethoxy-2-nitroveratrole carbamate can be used. Examples of these amino acids are nitrobenzyl protected lysine, nitrobenzyl protected cysteine, and 3,5-dimethoxy-2-nitrobenzyl protected diaminopropionic acid. Other reactive amino acids include, for example, halogenated phenyalanine derivatives, an unnatural amino acid containing an azide moiety, an unnatural amino acid containing an acetylene moiety, or an unnatural amino acid containing an acetyl group. Examples of reactive unnatural amino acids include 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, p-azido-phenylalanine, o-azido-phenylalanine, 2-amino-2-(4-(ethynyloxy)phenyl)acetic acid, p-acetyl-phenylalanine, p-ethynyl-phenylalanine, 2-amino-4-oxopentanoic acid, and 2-amino-5-oxohexanoic acid. Reactive unnatural amino acids that include acetyl groups can be coupled to fluorescent moieties containing a hydrazide. Unnatural amino acids containing azide or acetylene moieties can be coupled to fluorescent moieties using “click” chemistry (e.g., involving a 3+2 cycloaddition reaction).

Typically, the unnatural amino acids of the invention are selected or designed to provide additional characteristics unavailable in the twenty-one natural amino acids. For example, unnatural amino acid are optionally designed or selected to modify the biological properties of a protein, e.g., a protein into which they are incorporated. For example, the following properties are optionally modified by inclusion of an unnatural amino acid into a protein: toxicity, biodistribution, solubility, stability, e.g., thermal, hydrolytic, oxidative, resistance to enzymatic degradation, and the like, facility of purification and processing, structural properties, spectroscopic properties, chemical and/or photochemical properties, catalytic activity, redox potential, half-life, and ability to react with other molecules, e.g., covalently or noncovalently.

Proteins Comprising Unnatural Amino Acids

The incorporation of an unnatural amino acid into a protein can be performed in order to tailor changes in protein structure and/or function, e.g., to change the size, acidity, nucleophilicity, hydrogen bonding, hydrophobicity, or accessibility of protease target sites. Proteins that include an unnatural amino acid can have enhanced or even entirely new catalytic or physical properties. For example, the following properties are optionally modified by inclusion of an unnatural amino acid into a protein: toxicity, biodistribution, structural properties, spectroscopic properties, chemical and/or photochemical properties, catalytic ability, half-life (e.g., serum half-life), and the ability to react with other molecules, e.g., covalently or noncovalently. The compositions including proteins that include at least one unnatural amino acid are useful for, e.g., novel therapeutics, diagnostics, catalytic enzymes, binding proteins (e.g., antibodies), and the study of protein structure and function.

In another example, unnatural amino acids can be incorporated into GABA_{A} ion channels (e.g., α2β2γ3) to obtain high-precision structural and functional information about the protein. The mutated protein can be used to determine the details of how compounds bind to the GABA_{A} ion channel. The unnatural amino acids that can be used for this purpose include such molecules as thienylalanine, pyrrole alanine, N_{T}-methyl-histidine, 2-amino-5-oxohexanoic acid, norvaline, norleucine, and phenyalanine derivatives like 3,5-F₂-phenyalanine, cyclohexyalanine, and 4-Cl-phenyalanine.

Unnatural amino acids can also be incorporated into proteins in order to be able to tether compounds, such as peptides or toxic moieties, to proteins via a reactive unnatural amino acid. Unnatural amino acids which can be used for this purpose include, for example, molecules with azido groups attached such as p-azido-phenylalanine and o-azido-phenylalanine; allyl containing unnatural amino acids; and keto derivatives of phenylalanine and other natural amino acids such as 2-amino-4-pentanoic acid and 2-amino-5-oxohexanoic acid.

A composition produced by the present method can further include at least one protein with at least one, e.g., at least two, at least three, at least four, at least five, at least six, at least seven, at least eight, at least nine, at least ten, or more unnatural amino acids. For a given protein with more than one unnatural amino acid, the unnatural amino acids can be identical or different (e.g., the protein can include two or more different types of unnatural amino acids, or can include two or more different sites having unnatural amino acids, or both).

A large number of different proteins can be made using the present methods. For example, therapeutic proteins incorporating an unnatural amino acid can be produced. Examples of therapeutic and other proteins that can be modified to comprise one or more unnatural amino acids include, e.g., Alpha-1 antitrypsin, Angiostatin, Antihemolytic factor, antibodies, Apolipoprotein, Apoprotein, Atrial natriuretic factor, Atrial natriuretic polypeptide, Atrial peptides, C—X—C chemokines (e.g., T39765, NAP-2, ENA-78, Gro-a, Gro-b, Gro-c, IP-10, GCP-2, NAP-4, SDF-1, PF4, MIG), Calcitonin, CC chemokines (e.g., Monocyte chemoattractant protein-1, Monocyte chemoattractant protein-2, Monocyte chemoattractant protein-3, Monocyte inflammatory protein-1 alpha, Monocyte inflammatory protein-i beta, RANTES, 1309, R83915, R91733, HCC1, T58847, D31065, T64262), CD40 ligand, C-kit Ligand, Collagen, Colony stimulating factor (CSF), Complement factor 5a, Complement inhibitor, Complement receptor 1, cytokines, (e.g., epithelial Neutrophil Activating Peptide-78, GROα/MGSA, GROβ, GROγ, MIP-1α, MIP-16, MCP-1), Epidermal Growth Factor (EGF), Erythropoietin, Exfoliating toxins A and B, Factor IX, Factor VII, Factor VIII, Factor X, Fibroblast Growth Factor (FGF), Fibrinogen, Fibronectin, G-CSF, GM-CSF, Glucocerebrosidase, Gonadotropin, growth factors, Hedgehog proteins (e.g., Sonic, Indian, Desert), Hemoglobin, Hepatocyte Growth Factor (HGF), Hirudin, Human serum albumin, Insulin, Insulin-like Growth Factor (IGF), interferons (e.g., IFN-.alpha., IFN-β, IFN-.gamma.), interleukins (e.g., IL-1, IL-2, IL-3, IL-4, IL-5, IL-6, IL-7, IL-8, IL-9, IL-10, IL-11, IL-12, etc.), Keratinocyte Growth Factor (KGF), Lactoferrin, leukemia inhibitory factor, Luciferase, Neurturin, Neutrophil inhibitory factor (NIF), oncostatin M, Osteogenic protein, Parathyroid hormone, PD-ECSF, PDGF, peptide hormones (e.g., Human Growth Hormone), Pleiotropin, Protein A, Protein G, Pyrogenic exotoxins A, B, and C, Relaxin, Renin, SCF, Soluble complement receptor I, Soluble I-CAM 1, Soluble interleukin receptors (IL-1, 2, 3, 4, 5, 6, 7, 9, 10, 11, 12, 13, 14, 15), Soluble TNF receptor, Somatomedin, Somatostatin, Somatotropin, Streptokinase, Superantigens, i.e., Staphylococcal enterotoxins (SEA, SEB, SEC1, SEC2, SEC3, SED, SEE), Superoxide dismutase, Toxic shock syndrome toxin (TSST-1), Thymosin alpha 1, Tissue plasminogen activator, Tumor necrosis factor beta (TNF beta), Tumor necrosis factor receptor (TNFR), Tumor necrosis factor-alpha (TNF alpha), Vascular Endothelial Growth Factor (VEGEF), and Urokinase. The amino acid and corresponding nucleic acid sequences coding for many of these proteins and variants thereof are known (see, e.g., Genbank).

A variety of enzymes (e.g., industrial enzymes or enzymes of involved in disease states), such as oxidoreductases, transferases, hydrolases, lyases, isomerases, and ligases, can also be modified to include one or more unnatural amino acid according to the methods herein. Such enzymes include, e.g., amidases, amino acid racemases, acylases, dehalogenases, dioxygenases, diarylpropane peroxidases, epimerases, epoxide hydrolases, esterases, isomerases, kinases, glucose isomerases, glycosidases, glycosyl transferases, haloperoxidases, monooxygenases (e.g., p450s), lipases, lignin peroxidases, nitrile hydratases, nitrilases, proteases, phosphatases, subtilisins, transaminase, nucleases, kinases, ATPases, phosphatases, phosphodiesterases, lipases, and proteases.

Source and Host Organisms

RS-tRNA pairs from the following organisms have been found to be orthogonal to eukaryotic cells, and therefore to be useful in the present systems and methods: L. lactis, Gluconobacter oxydans and Rhodospirullum rubrum. Host cells can be from any of the eukaryotic groups, including cells from organisms which belong to a taxonomic kingdom selected from the group consisting of Animalia and Fungi. For example, a host cell can be yeast cell such as S. cerevisiae, or can be a member of a taxonomic class selected from the group consisting of Mammalia and Amphibia. Particularly preferred cells for use as hosts in the present methods and systems include CHO cells, BHK cells, and human cells such as HEK cells.

The incorporation of unnatural amino acids in vivo can be done without significant perturbation of the host. For example, because the suppression efficiency for the UAG codon depends upon the competition between the O-tRNA, e.g., the amber suppressor tRNA, and release factor 1 (RF1) (which binds to the UAG codon and initiates release of the growing peptide from the ribosome), the suppression efficiency can be modulated by, e.g., either increasing the expression level of O-tRNA, e.g., the suppressor tRNA, or using an RF1 deficient strain.

Methods of Producing O-RS/O-tRNA Pairs

One strategy for generating an orthogonal tRNA/synthetase pair involves importing a tRNA/synthetase pair from another organism into the host cell. The properties of the heterologous synthetase candidate include, e.g., that it does not charge any host cell tRNA, and the properties of the heterologous tRNA candidate include, e.g., that it is not acylated by any host cell synthetase. In addition, the suppressor tRNA derived from the heterologous tRNA is orthogonal to all host cell synthetases.

A similar approach involves the use of a heterologous synthetase as the orthogonal synthetase but a mutant initiator tRNA of the same organism or a related organism as the orthogonal tRNA. RajBhandary and coworkers found that an amber mutant of human initiator tRNAfMet is acylated by E. coli GlnRS and acts as an amber suppressor in yeast cells only when EcGlnRS is coexpressed [see, A. K. Kowal, C. Kohrer and U. L. RajBhandary, Proc. Natl. Acad. Sci. USA, 98:2268 (2001)]. This pair thus represents an orthogonal pair for use in yeast. Also, an E. coli initiator tRNAfMet amber mutant was found that is inactive toward any E. coli synthetases. A mutant yeast TyrRS was selected that charges this mutant tRNA, resulting in an orthogonal pair in E. coli [see, A. K. Kowal, et al, (2001), supra].

Positive and Negative Selection

An O-RS can be produced by generating a pool of mutant synthetases from the framework of a wild-type synthetase, and then selecting for mutated RS molecules based on their specificity for an unnatural amino acid relative to the common twenty natural amino acids. An orthogonal aminoacyl synthetase can be produced, for example, by mutating the synthetase, e.g., at the active site in the synthetase, at the editing mechanism site in the synthetase, and/or at different sites by combining different domains of synthetases, and applying a selection process. In one embodiment, an in vivo selection/screening strategy is used which is based on the combination of a positive selection step followed by a negative selection step. In the positive selection, suppression of the selector codon introduced at a nonessential position or positions of a positive marker allows cells to survive under positive selection pressure. In the presence of both natural and unnatural amino acids, survivors thus encode active synthetases charging the orthogonal suppressor tRNA with either a natural or unnatural amino acid. In the negative selection, suppression of a selector codon introduced at a nonessential position or positions of a negative marker removes synthetases with natural amino acid specificities. Survivors of the negative and positive selection steps encode synthetases that aminoacylate (charge) the orthogonal suppressor tRNA with unnatural amino acids only. These synthetases can then be subjected to further mutagenesis, e.g., DNA shuffling or other recursive mutagenesis methods, for example to allow them to be expressed efficiently in a host cell. These steps can be carried out in different orders in order to identify O-RS/O-tRNA pairs, such as by employing a negative selection/screening followed by positive selection/screening or further combinations thereof.

For example, a selector codon, e.g., an amber codon, can be placed in a reporter gene, e.g., an antibiotic resistance gene such as β-lactamase, with a selector codon, e.g., TAG. This construct is placed in an expression vector with members of a mutated RS library. This expression vector along with an expression vector with an orthogonal tRNA, e.g., a orthogonal suppressor tRNA, are introduced into a cell, which is grown in the presence of a selection agent, e.g., antibiotic media, such as ampicillin. Only if the synthetase is capable of aminoacylating (charging) the suppressor tRNA with some amino acid does the selector codon get decoded, allowing survival of the cell on antibiotic media.

Applying this selection in the presence of the unnatural amino acid, the synthetase genes that encode synthetases that have some ability to aminoacylate are selected away from those synthetases that have no activity. The resulting pool of synthetases can be charging any of the 20 naturally occurring amino acids or the unnatural amino acid. To further select for those synthetases that exclusively charge the unnatural amino acid, a second selection, e.g., a negative selection can be applied. In this case, an expression vector containing a negative selection marker and an O-tRNA is used, along with an expression vector containing a member of the mutated RS library. This negative selection marker contains at least one selector codon, e.g., TAG. These expression vectors are introduced into another cell and grown without unnatural amino acids and, optionally, a selection agent, e.g., tetracycline. In the negative selection, those synthetases with specificities for natural amino acids charge the orthogonal tRNA, resulting in suppression of a selector codon in the negative marker and cell death. Since no unnatural amino acid is added, synthetases with specificities for the unnatural amino acid survive. For example, a selector codon, e.g., a stop codon, is introduced into the reporter gene, e.g., a gene that encodes a toxic protein, such as barnase. If the synthetase is able to charge the suppressor tRNA in the absence of unnatural amino acid, the cell will be killed by translating the toxic gene product. Survivors passing both selection/screens encode synthetases specifically charge the orthogonal tRNA with an unnatural amino acid.

In another embodiment, the positive selection step can include: introducing a positive selection marker, e.g., an antibiotic resistance gene, and a library of mutant RS molecules into a plurality of cells, wherein the positive selection marker comprises at least one selector codon, e.g., an amber codon; growing the plurality of cells in the presence of a selection agent; and selecting cells that survive in the presence of the selection agent by suppressing the at least one selector codon in the positive selection marker, thereby providing a subset of positively selected cells that contains the pool of active mutant RS molecules. Optionally, the selection agent concentration can be varied.

Positive selection can also be based on suppression of a selector codon in a positive selection marker, e.g., a chloramphenicol acetyltransferase (CAT) gene comprising a selector codon, e.g., an amber stop codon, in the CAT gene, so that chloramphenicol can be applied as the positive selection pressure. In addition, the CAT gene can be used as both a positive marker and negative marker in the presence and absence of unnatural amino acid. Optionally, the CAT gene comprising a selector codon can be used for the positive selection and a negative selection marker, e.g., a toxic marker, such as a barnase gene comprising at least one or more selector codons, is used for the negative selection.

The steps used in selection can include, e.g., a direct replica plate method. For example, after passing the positive selection, cells can be grown in the presence of either ampicillin or chloramphenicol and the absence of the unnatural amino acid. Those cells that do not survive are isolated from a replica plate supplemented with the unnatural amino acid. No transformation into a second negative selection strain is needed, and the phenotype is known. Compared to other potential selection markers, a positive selection based on antibiotic resistance offers the ability to tune selection stringency by varying the concentration of the antibiotic, and to compare the suppression efficiency by monitoring the highest antibiotic concentration cells can survive. In addition, the growth process is also an enrichment procedure. This can lead to a quick accumulation of the desired phenotype.

In another embodiment, negatively selecting the pool of candidates for active mutant RS molecules includes: isolating the pool of active mutant RS molecules from a positive selection step; introducing a negative selection marker, where the negative selection marker is a toxic marker gene, e.g., a ribonuclease barnase gene, comprising at least one selector codon, and the pool of active mutant RS molecules into a plurality of cells of a second organism; and then selecting cells that survive in a first media not supplemented with the unnatural amino acid, but fail to survive in a second media supplemented with the unnatural amino acid, thereby providing surviving cells with at least one recombinant O-RS, which is specific for the unnatural amino acid. Optionally, the negative selection marker can comprise two or more selector codons.

In a further aspect, positive selection can be based on suppression of a selector codon at a nonessential position in the β-lactamase gene, rendering cells ampicillin resistant; and a negative selection using the ribonuclease bamase as the negative marker can be used. In contrast to β-lactamase, which is secreted into the periplasm, CAT localizes in the cytoplasm; moreover, ampicillin is bacteriocidal, while chloramphenicol is bacteriostatic.

The stringency of the selection steps, e.g., the positive selection step, the negative selection step or both the positive and negative selection steps in the above described-methods, optionally include varying the selection stringency. For example, because bamase is an extremely toxic protein, the stringency of the negative selection can be controlled by introducing different numbers of selector codons into the barnase gene. In one aspect of the present invention, the stringency is varied because the desired activity can be low during early rounds. Thus, less stringent selection criteria can be applied in early rounds and more stringent criteria can be applied in later rounds of selection.

Generating O-RS/O-tRNA Pairs from Libraries

In one embodiment, orthogonal aminoacyl-tRNA synthetases can be generated recombinantly. Methods for producing a recombinant O-RS include: (a) generating a library of mutant RS molecules derived from at least one aminoacyl-tRNA synthetase (RS) from a first organism, e.g., L. lactis; (b) selecting the library of mutant RS molecules for members that aminoacylate an orthogonal tRNA (O-tRNA) in the presence of an unnatural amino acid and a natural amino acid, thereby providing a pool of active mutant RS molecules; and, (c) negatively selecting the pool for active mutant RS molecules that preferentially aminoacylate the O-tRNA in the absence of the unnatural amino acid, thereby providing the at least one recombinant O-RS. The recombinant O-RS molecules produced in this way preferentially aminoacylate the O-tRNA with the unnatural amino acid. Optionally, more mutations can be introduced by mutagenesis, e.g., random mutagenesis, recombination or the like, into the selected synthetase genes to generate a second-generation synthetase library, which is used for further rounds of selection until a mutant synthetase with desired activity is evolved. Orthogonal tRNA/synthetase pairs can also optionally be generated by importing such pairs from a first organism into a second organism.

The library of mutant RS molecules can be generated using various mutagenesis techniques known in the art. For example, the mutant RS molecules can be generated by site-specific mutations, random point mutations, in vitro homologous recombination, or chimeric constructs. A chimeric library can screened for a variety of properties, e.g., for members that are expressed and in frame, for members that lack activity with a desired synthetase, and/or for members that show activity with a desired synthetase.

In one embodiment, mutations can be introduced into the editing site of the synthetase to hamper the editing mechanism and/or to alter substrate specificity. Libraries of mutant RS molecules can also include chimeric synthetase libraries. It should be noted that libraries of tRNA synthetases from various organisms (e.g., microorganisms such as eubacteria or archaebacteria), as well as libraries comprising natural diversity (such as libraries that comprise natural diversity (see, e.g., U.S. Pat. No. 6,238,884 to Short et al. and references therein, U.S. Pat. No. 5,756,316 to Schallenberger et al; U.S. Pat. No. 5,783,431 to Petersen et al; U.S. Pat. No. 5,824,485 to Thompson et al; and U.S. Pat. No. 5,958,672 to Short et al), can optionally be constructed and screened for orthogonal RS/tRNA pairs.

Selection Strategy Alternatives

Other types of selections can also be used to produce O-RS, O-tRNA, and O-tRNA/O-RS pairs. In one embodiment, the positive selection step, the negative selection step, or both the positive and negative selection steps described above can include using a reporter detected by fluorescence-activated cell sorting (FACS). For example, a positive selection can be done first with a positive selection marker, e.g., chloramphenicol acetyltransferase (CAT) gene, where the CAT gene comprises a selector codon, e.g., an amber stop codon, in the CAT gene, which is followed by a negative selection screen based on the inability to suppress a selector codon(s), e.g., two or more, at positions within a negative marker, e.g., T7 RNA polymerase gene. In another embodiment, the positive selection marker and the negative selection marker can be found on the same vector, e.g., a plasmid. Expression of the negative marker drives expression of the reporter, e.g., green fluorescent protein (GFP). The stringency of the selection and screen can be varied, e.g., the intensity of the light need to fluorescence the reporter can be varied. In another embodiment, a positive selection can be done with a reporter as a positive selection marker screened by FACs, followed by a negative selection screen based on the inability to suppress a selector codon at positions within a negative marker, e.g., bamase gene.

Optionally, the reporter is displayed on a cell surface, e.g., in a phage display system. Cell-surface display, such as the OmpA-based cell-surface display system, relies on the expression of a particular epitope, e.g., a poliovirus C3 peptide fused to an outer membrane porin OmpA, on the surface of an E. coli cell [see, Francisco, J. A., Campbell, R., Iverson, B. L. & Georgoiu, G. Production and fluorescence-activated cell sorting of E. coli expressing a functional antibody fragment on the external surface. Proc. Natl. Acad. Sci. USA 90:10444-8 (1993)]. The epitope is displayed on the cell surface only when a selector codon in the protein message is suppressed during translation. The displayed peptide then contains the amino acid recognized by one of the mutant aminoacyl-tRNA synthetases in the library, and the cell containing the corresponding synthetase gene can be isolated with antibodies raised against peptides containing specific unnatural amino acids.

Methods for generating specific O-tRNA/O-RS pairs further include: (a) generating a library of mutant tRNAs derived from at least one tRNA from a first organism; (b) negatively selecting the library for mutant tRNAs that are aminoacylated by an aminoacyl-tRNA synthetase (RS) from a second organism in the absence of a RS from the first organism, thereby providing a pool of mutant tRNAs; and (c) selecting the pool of mutant tRNAs for members that are aminoacylated by an introduced orthogonal RS(O-RS), thereby providing at least one recombinant O-tRNA. The at least one recombinant O-tRNA recognizes a selector codon and is not efficiency recognized by the RS from the second organism and is preferentially aminoacylated by the O-RS. The method also includes: (d) generating a library of mutant RS molecules derived from at least one aminoacyl-tRNA synthetase (RS) from a third organism; (e) selecting the library of mutant RS molecules for members that preferentially aminoacylate the at least one recombinant O-tRNA in the presence of an unnatural amino acid and a natural amino acid, thereby providing a pool of active mutant RS molecules; and (f) negatively selecting the pool for active mutant RS molecules that preferentially aminoacylate the at least one recombinant O-tRNA in the absence of the unnatural amino acid, thereby providing the at least one specific O-tRNA/O-RS pair, where the at least one specific O-tRNA/O-RS pair comprises at least one recombinant O-RS that is specific for the unnatural amino acid and the at least one recombinant O-tRNA. Pairs produced by the methods of the present invention are also included.

Methods of Producing Proteins Having Unnatural Amino Acids

The present methods of specifically incorporating an unnatural amino acid into a protein are preferably carried out in vivo in a cell. The O-tRNA/O-RS pairs or individual components of the present invention can then be used with a host system's translation machinery, which results in an unnatural amino acid being incorporated into a protein. For example, when an O-tRNA/O-RS pair is introduced into a host, e.g., CHO cells, the pair leads to the in vivo incorporation of an unnatural amino acid, e.g., a synthetic amino acid, such as O-methyl-L-tyrosine, which can be exogenously added to the growth medium, into a protein, e.g., dihydrofolate reductase or a therapeutic protein such as EPO, in response to a selector codon, e.g., an amber nonsense codon.

Alternatively, the present compositions can be used with an in vitro translation system to produce proteins. In such embodiments, the translation components of a particular organism, such as an insect (i.e. from an insect cell line such as the Sf9 cell line, available from Orbigen, Inc., San Diego, Calif.) are combined in vitro with one or more unnatural amino acids, one or more O-tRNA/O-RS pairs, and other components required to produce a protein. O-tRNA/O-RS pairs in this embodiment can be produced recombinantly.

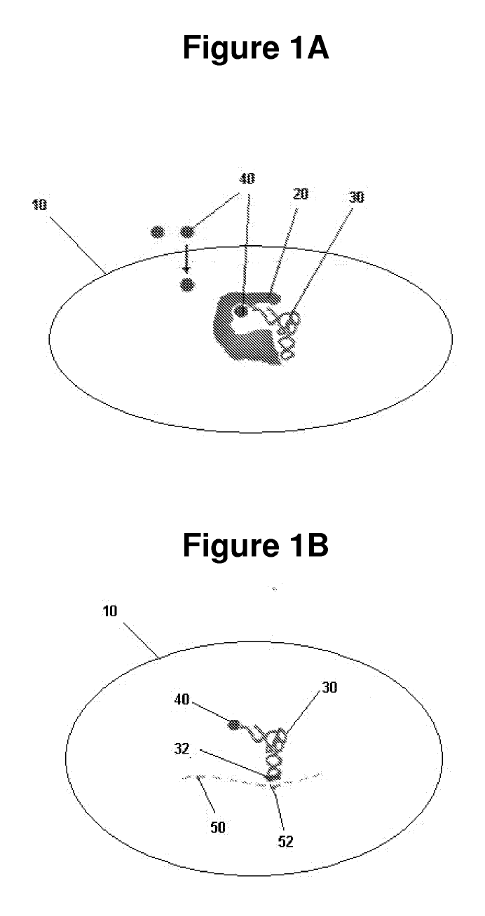

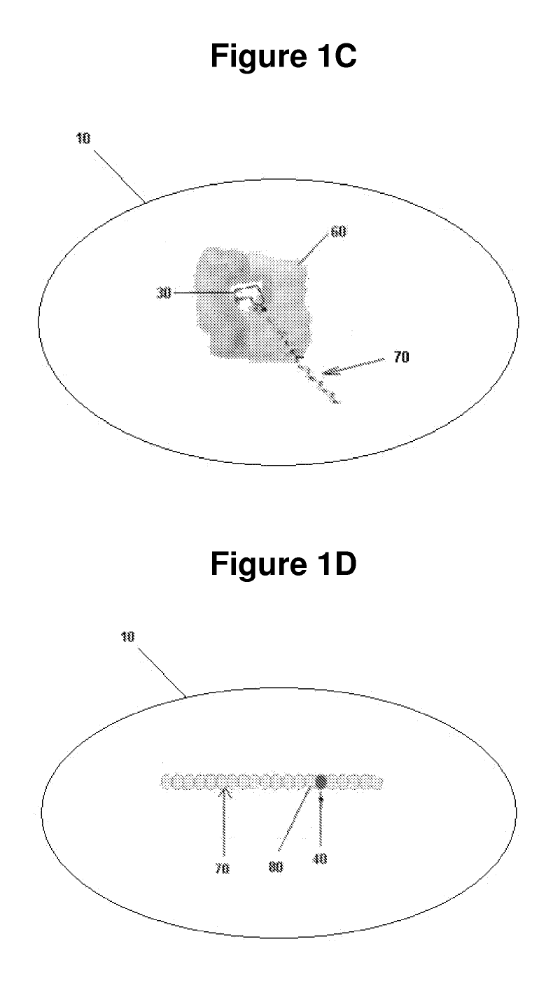

The site-specific incorporation of unnatural amino acids into proteins in vivo according to the present methods is schematically illustrated in FIG. 1. A cell 10 is provided with an aminoacyl synthetase derived from L. lactis 20 and tRNA derived from L. lactis 30, as described more fully below. The synthetase 20 aminoacylates the tRNA 30 with an unnatural amino acid 40 which is introduced into the cell 10.

The cell 10 further comprises an mRNA molecule 50 having a selector codon 52. When a ribosome 60 encounters the selector codon 52 in the process of translating the mRNA molecule 50, the anticodon 32 of the tRNA 30 recognizes the selector codon 52 and the ribosome 60 catalyzes the formation of a peptide bond between the unnatural amino acid 40 and a natural amino acid 80 adjacent to it in the peptide chain of the protein 70 being formed. A full-length protein product is thus produced which includes the unnatural amino acid 40 incorporated therein.

Cellular Uptake of Unnatural Amino Acids

An unnatural amino acid must be taken up or otherwise transported into a cell in order for it to be incorporated into a protein in vivo. In order to determine whether a particular unnatural amino acid can be taken up by a particular cell type, a rapid screen can be performed to assess whether it will be taken up by such cells.

To screen for the potential toxicity of an unnatural amino acid, a screen in minimal media can be performed. Toxicities are typically sorted into five groups: (1) no toxicity, in which no significant change in cell doubling times occurs; (2) low toxicity, in which doubling times increase by less than about 10%; (3) moderate toxicity, in which doubling times increase by about 10% to about 50%; (4) high toxicity, in which doubling times increase by about 50% to about 100%; and (5) extreme toxicity, in which doubling times increase by more than about 100% [see, e.g., Liu, D. R. & Schultz, P. G. Progress toward the evolution of an organism with an expanded genetic code, Proceedings of the National Academy of Sciences of the U.S.A., 96:4780-4785 (1999)]. The toxicity of the amino acids scoring as highly or extremely toxic are typically measured as a function of their concentration to obtain IC₅₀ values.

To identify possible uptake pathways for toxic amino acids, toxicity assays are optionally repeated at IC₅₀ levels, e.g., in media supplemented with an excess of a structurally similar natural amino acid. For toxic amino acids, the presence of excess natural amino acid typically rescues the ability of the cells to grow in the presence of the toxin, presumably because the natural amino acid effectively outcompetes the toxin for either cellular uptake or for binding to essential enzymes. In these cases, the toxic amino acid is optionally assigned a possible uptake pathway and labeled a “lethal allele” whose complementation is required for cell survival. These lethal alleles are extremely useful for assaying the ability of cells to uptake nontoxic unnatural amino acids. Complementation of the toxic allele, evidenced by the restoration of cell growth, suggests that the nontoxic amino acid is taken up by the cell, possibly by the same uptake pathway as that assigned to the lethal allele. A lack of complementation is inconclusive.

Unnatural amino acids can also be transported into a cell independent of an amino acid uptake pathway, for example, through the use of peptide permeases, which transport dipeptides and tripeptides across a cytoplasmic membrane. Peptide permeases are not very side-chain specific, and the KD values for their substrates are comparable to KD values of amino acid permeases, e.g., about 0.1 mM to about 10 mM [see, e.g., Nickitenko, A., Trakhanov, S. & Quiocho, S, A structure of DppA, a periplasmic depeptide transport/chemosensory receptor, Biochemistry, 34:16585-16595 (1995) and Dunten, P., Mowbray, S. L., Crystal structure of the dipeptide binding protein from E. coli involved in active transport and chemotaxis, Protein Science, 4:2327-34 (1995)]. The unnatural amino acids are taken up as conjugates of natural amino acids, such as lysine, and released into the cytoplasm upon hydrolysis of the dipeptide by an endogenous peptidase.

Alternatively, in some cases amino acids can be produced by biosynthetic pathways in vivo. Pathways for producing unnatural amino acids in this way are optionally generated by expressing new enzymes in a host cell or modifying existing pathways. For example, recursive recombination, e.g., as developed by Maxygen, Inc., can be used to develop novel enzymes and pathways [see, e.g., Stemmer 1994, “Rapid evolution of a protein in vitro by DNA shuffling,” Nature, Vol. 370 No. 4: Pg. 389-391; Stemmer, “DNA shuffling by random fragmentation and reassembly: In vitro recombination for molecular evolution,” Proc. Natl. Acad. Sci. USA, Vol. 91:10747-10751(1994)]. Similarly DesignPath™, developed by Genencor can be used for metabolic pathway engineering, e.g., to engineer a pathway to create O-methyl-L-trosine in a host cell.

Typically, the unnatural amino acid produced with an engineered biosynthetic pathway of the present invention is produced in a concentration sufficient for efficient protein biosynthesis, e.g., a natural cellular amount, but not to such a degree as to affect the concentration of other amino acids in a host cell or exhaust cellular resources. Typical concentrations produced in vivo in this manner are about 10 mM to about 0.05 mM. Once a host cell is transformed with a plasmid comprising the genes used to produce enzymes desired for a specific pathway and a twenty-first amino acid (e.g., pAF, dopa, or O-methyl-L-tyrosine) is generated, in vivo selections are optionally used to further optimize the production of the unnatural amino acid for both ribosomal protein synthesis and cell growth.

For example, plant O-methyltransferases, which convert a hydroxyl group into a methoxyl group can be expressed in a cell (such as an animal cell) which lacks this endogenous enzyme. Examples of such enzymes include (iso)eugenol O-methyltransferase (IEMT) and caffeic acid O-methyltransferase (COMT). IEMT methylates eugenol/isoeugenol, and COMT methylates caffeic acid. The substrates of these two enzymes are similar to tyrosine. A combinatorial approach can be used to evolve the substrate specificity of both enzymes to tyrosine, thereby converting tyrosine to O-methyl-L-tyrosine.

Alternatively, to produce the unnatural amino acid p-aminophenylalanine (pAF) in vivo, genes relied on in the pathways leading to chloramphenicol and pristinamycin can be used. For example, in Streptomyces venezuelae and Streptomyces pristinaespiralis, these genes produce pAF as a metabolic intermediate [see, e.g., Blanc, V., et al., Identification and analysis of genes from S. pristinaespiralis encoding enzymes involved in the biosynthesis of the 4-dimethylamino-L-phenylalanine precursor of pristinamycin I, Molecular Microbiology, 23(2):191-202 (1997)]. The unnatural amino acid pAF can alternatively be synthesized from chorismate, which is a biosynthetic intermediate in the synthesis of aromatic amino acids in some organisms (such as E. coli). To synthesize pAF from chorismate, a cell typically uses a chorismate synthase, a chorismate mutase, a dehydrogenase (such as a prephenate dehydrogense), and an amino transferase. For example, using the S. venezuelae enzymes PapA, PapB, and PapC together with an E. coli aminotransferase, chorismate can be used to produce pAF.

A plasmid for use in the biosynthesis of pAF can comprise, for example, the S. venezuelae genes papA, papB, and papC cloned into a plasmid, under control of, e.g., a lac or lpp promotor. The plasmid is used to transform a cell, e.g., a eukaryotic cell, such that the cell produces the enzymes encoded by the genes. When expressed, the enzymes catalyze one or more reactions designed to produce a desired unnatural amino acid, e.g., pAF.

General Techniques

General texts which describe molecular biological techniques applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”)). These texts describe mutagenesis, the use of vectors, promoters and many other relevant topics related to, e.g., the generation of orthogonal tRNA, orthogonal synthetases, and pairs thereof.

Various types of mutagenesis can be used in the present invention, e.g., to produce novel sythetases or tRNAs. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like. Mutagenesis, e.g., involving chimeric constructs, are also included in the present invention. In one embodiment, mutagenesis can be guided by known information of the naturally occurring molecule or altered or mutated naturally occurring molecule, e.g., sequence, sequence comparisons, physical properties, crystal structure or the like.

The above texts and examples found herein describe these procedures as well as the following publications and references cited within: Sieber, et al., Nature Biotechnology, 19:456460 (2001); Ling et al., Approaches to DNA mutagenesis: an overview, Anal Biochem. 254(2): 157-178 (1997); Dale et al., Oligonucleotide-directed random mutagenesis using the phosphorothioate method, Methods Mol. Biol. 57:369-374 (1996); I. A. Lorimer, I. Pastan, Nucleic Acids Res. 23, 3067-8 (1995); W. P. C. Stemmer, Nature 370, 389-91 (1994); Arnold, Protein engineering for unusual environments, Current Opinion in Biotechnology 4:450-455 (1993); Bass et al., Mutant Trp repressors with new DNA-binding specificities, Science 242:240-245 (1988); Fritz et al., Oligonucleotide-directed construction of mutations: a gapped duplex DNA procedure without enzymatic reactions in vitro, Nucl. Acids Res. 16: 6987-6999 (1988); Kramer et al., Improved enzymatic in vitro reactions in the gapped duplex DNA approach to oligonucleotide-directed construction of mutations, Nucl. Acids Res. 16: 7207 (1988); Sakamar and Khorana, Total synthesis and expression of a gene for the .alpha.-subunit of bovine rod outer segment guanine nucleotide-binding protein (transducin), Nucl. Acids Res. 14: 6361-6372 (1988); Sayers et al., Y-T Exonucleases in phosphorothioate-based oligonucleotide-directed mutagenesis, Nucl. Acids Res. 16:791-802 (1988); Sayers et al., Strand specific cleavage of phosphorothioate-containing DNA by reaction with restriction endonucleases in the presence of ethidium bromide, (1988) Nucl. Acids Res. 16: 803-814; Carter, Improved oligonucleotide-directed mutagenesis using M13 vectors, Methods in Enzymol. 154: 382403 (1987); Kramer & Fritz Oligonucleotide-directed construction of mutations via gapped duplex DNA, Methods in Enzymol. 154:350-367 (1987); Kunkel, The efficiency of oligonucleotide directed mutagenesis, in Nucleic Acids & Molecular Biology (Eckstein, F. and Lilley, D. M. J. eds., Springer Verlag, Berlin)) (1987); Kunkel et al., Rapid and efficient site-specific mutagenesis without phenotypic selection, Methods in Enzymol. 154, 367-382 (1987); Zoller & Smith, Oligonucleotide-directed mutagenesis: a simple method using two oligonucleotide primers and a single-stranded DNA template, Methods in Enzymol. 154:329-350 (1987); Carter, Site-directed mutagenesis, Biochem. J. 237:1-7 (1986); Eghtedarzadeh & Henikoff, Use of oligonucleotides to generate large deletions, Nucl. Acids Res. 14: 5115 (1986); Mandecki, Oligonucleotide-directed double-strand break repair in plasmids of E. coli: a method for site-specific mutagenesis, Proc. Natl. Acad. Sci. USA, 83:7177-7181 (1986); Naka & Eckstein, Inhibition of restriction endonuclease Nci I cleavage by phosphorothioate groups and its application to oligonucleotide-directed mutagenesis, Nucl. Acids Res. 14: 9679-9698 (1986); Wells et al., Importance of hydrogen-bond formation in stabilizing the transition state of subtilisin, Phil. Trans. R. Soc. Lond. A 317: 415423 (1986); Botstein & Shortle, Strategies and applications of in vitro mutagenesis, Science 229:1193-1201 (1985); Carter et al., Improved oligonucleotide site-directed mutagenesis using M13 vectors, Nucl. Acids Res. 13: 4431-413 (1985); Grundstrom et al., Oligonucleotide-directed mutagenesis by microscale ‘shot-gun’ gene synthesis, Nucl. Acids Res. 13: 3305-3316 (1985); Kunkel, Rapid and efficient site-specific mutagenesis without phenotypic selection, Proc. Natl. Acad. Sci. USA 82:488492 (1985); Smith, In vitro mutagenesis, Ann. Rev. Genet. 19:423462 (1985); Taylor et al., The use of phosphorothioate-modified DNA in restriction enzyme reactions to prepare nicked DNA, Nucl. Acids Res. 13: 8749-8764 (1985); Taylor et al., The rapid generation of oligonucleotide-directed mutations at high frequency using phosphorothioate-modified DNA, Nucl. Acids Res. 13: 8765-8787 (1985); Wells et al., Cassette mutagenesis: an efficient method for generation of multiple mutations at defined sites, Gene 34:315-323 (1985); Kramer et al., The gapped duplex DNA approach to oligonucleotide-directed mutation construction, Nucl. Acids Res. 12: 9441-9456 (1984); Kramer et al., Point Mismatch Repair, Cell 38:879-887 (1984); Nambiar et al., Total synthesis and cloning of a gene coding for the ribonuclease S protein, Science 223: 1299-1301 (1984); Zoller & Smith, Oligonucleotide-directed mutagenesis of DNA fragments cloned into M13 vectors, Methods in Enzymol. 100:468-500 (1983); and Zoller & Smith, Oligonucleotide-directed mutagenesis using M13-derived vectors: an efficient and general procedure for the production of point mutations in any DNA fragment, Nucleic Acids Res. 10:6487-6500 (1982). Additional details on many of the above methods can be found in Methods in Enzymology Volume 154, which also describes useful controls for trouble-shooting problems with various mutagenesis methods.

Oligonucleotides, e.g., for use in mutagenesis or altering tRNAs, are typically synthesized chemically according to the solid phase phosphoramidite triester method described by Beaucage and Caruthers, Tetrahedron Letts. 22(20):1859-1862, (1981), e.g., using an automated synthesizer, as described in Needham-VanDevanter et al., Nucleic Acids Res., 12:6159-6168 (1984).

In addition, essentially any nucleic acid can be custom or standard ordered from any of a variety of commercial sources, such as The Midland Certified Reagent Company (Midland, Tex.), The Great American Gene Company (Pittsburgh, Pa.), ExpressGen Inc. (Chicago, Ill.), and Operon Technologies Inc. (Alameda, Calif.).

Other useful references, e.g. for cell isolation and culture (e.g., for subsequent nucleic acid isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds.) (1995) Plant Cell, Tissue and Organ Culture; Fundamental Methods Springer Lab Manual, Springer-Verlag (Berlin Heidelberg New York) and Atlas and Parks (eds.) The Handbook of Microbiological Media (1993) CRC Press, Boca Raton, Fla.

Several well-known methods of introducing target nucleic acids into bacterial cells are available, any of which can be used in the present invention. These include: fusion of the recipient cells with bacterial protoplasts containing the DNA, electroporation, projectile bombardment, and infection with viral vectors, etc. Bacterial cells can be used to amplify the number of plasmids containing DNA constructs of this invention. The bacteria are grown to log phase and the plasmids within the bacteria can be isolated by a variety of methods known in the art (see, for instance, Sambrook). In addition, a plethora of kits are commercially available for the purification of plasmids from bacteria, (see, e.g., EasyPrep™, FlexiPrep™, both from Pharmacia Biotech; StrataClean™, from Stratagene; and, QIAprep™ from Qiagen). The isolated and purified plasmids are then further manipulated to produce other plasmids, used to transfect cells or incorporated into related vectors to infect organisms. Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or preferably both. See, Giliman & Smith, Gene 8:81 (1979); Roberts, et al., Nature, 328:731 (1987); Schneider, B., et al., Protein Expr. Purif. 6435:10 (1995); Ausubel, Sambrook, Berger (all supra). A catalogue of Bacteria and Bacteriophages useful for cloning is provided, e.g., by the ATCC, e.g., The ATCC Catalogue of Bacteria and Bacteriophage (1992) Ghema et al. (eds.) published by the ATCC. Additional basic procedures for sequencing, cloning and other aspects of molecular biology and underlying theoretical considerations are also found in Watson et al. (1992) Recombinant DNA Second Edition Scientific American Books, NY.

### EXAMPLES

### Example 1

Identifying Orthogonal tRNA/RS Pairs and Hosts in Vitro

We prepared concentrated crude RS and total RNA from the following bacteria using published methods: Lactobacillus acidophilus, Lactobacillus casei, Lactococcus lactis, Gluconobacter oxydans and Rhodospirullum rubrum. As positive controls, we also isolated crude RS and total RNA from E. coli and Bacillus stearothermophilus (whose TyrRS/tRNA pairs have been shown to be orthogonal against mammalian TyrRS/tRNA pairs). We purchased bovine RS and total human tRNA.

Using the crude RS and RNA preparations, aminoacylation of Tyr tRNA was determined by measuring [³H]-Tyr incorporation in an in vitro assay. Reactions (60 μl) contained 50 mM Tris, 50 mM KCl, 2 mM DTT, 4 mM ATP, 2 mM Mg(OAc)₂, 0.3 nM [³H]-Tyr (54 ci/mmol), 10 μg total RNA preparation or 2 μg human tRNA and 25 μl concentrated crude bacterial RS preparation or 6-10 U bovine RS preparation. tRNA was omitted for control reactions. Following incubation at 37° C. for one hour, tRNA-[³H]-tyrosine was precipitated by transferring the reactions to tubes containing 3 ml ice cold 10% TCA and incubated on ice for one hour. The precipitates were collected by vacuum filtration on GF/C filters presoaked with 10% TCA. Filters were washed three times each with 1 ml 10% TCA and two times with 1 ml ice cold EtOH and air-dried. Filter-retained radioactivity was determined by liquid scintillation counting.

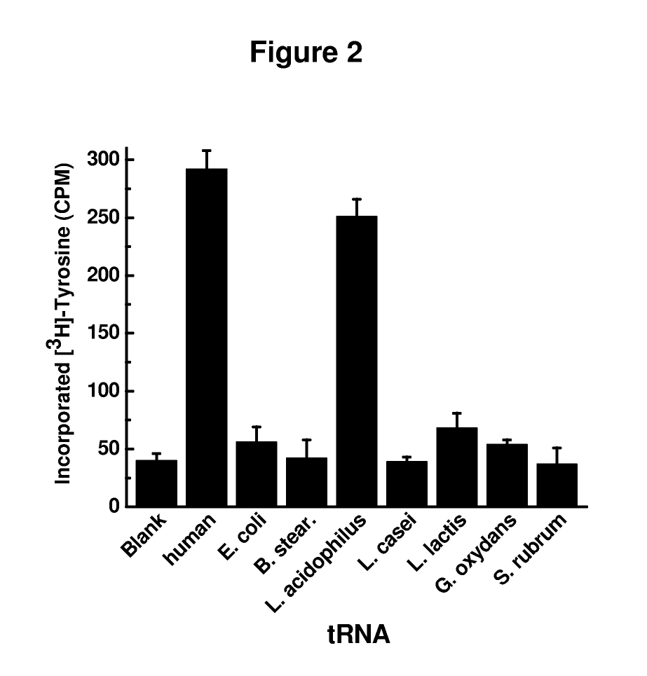

We first tested whether the bacterial Tyr tRNAs were orthogonal with respect to the bovine TyrRS (FIG. 2). The amount of radioactive TCA insoluble material collected on the filters represented tRNA-[³H]-Tyr. As expected, [³H]-Tyr incorporation above background was observed for reactions containing bovine RS and human RNA. As validation of our assay and consistent with published findings, we did not observe [³H]-Tyr incorporation in reactions containing E. coli and Bacillus stearothermophilus RNA. For the remaining bacteria RNA, with the exception of L. acidophilus, we did not observe any significant [³H]-Tyr incorporation above background. From these findings, we conclude that Tyr tRNA from L. casei, L. lactis, G. oxydans and R. rubrum are orthogonal with respect to mammalian TyrRS.

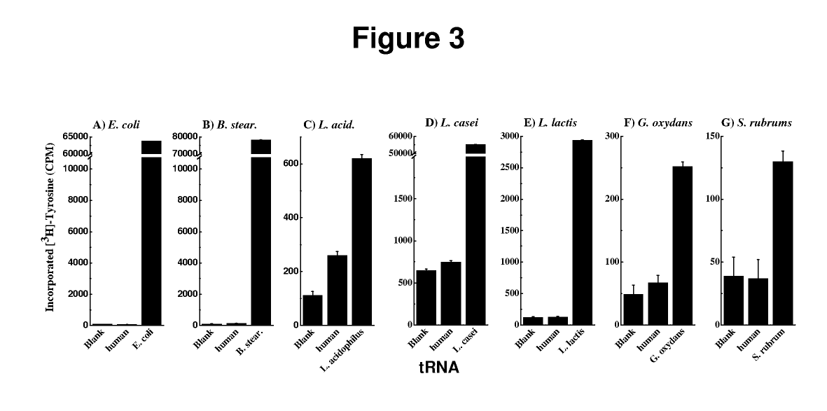

We next measured whether the bacterial TyrRS were orthogonal with respect to the bovine TyrRS. As validation of our assay and consistent with published findings, [³H]-Tyr incorporation was observed in reactions containing E. coli and B. stearothermophilus RS and their respective RNAs but not in reactions containing human RNA (FIG. 3, Panels A and B). For the remaining bacteria, with the exception of L. acidophilus and L. casei, we observed [³H]-Tyr incorporation above background only for reactions containing bacterial RS prep/bacterial RNA but not human RNA (FIG. 3, Panels C-G). From these findings, we conclude that TyrRS from L. lactis, G. oxydans and R. rubrum are orthogonal with respect to human tRNA.

### Example 2

Identifying O-RS/O-tRNA Pairs and Hosts in Vivo

E. coli cells are transformed with an expression vector containing a reporter gene, e.g., β-lactamase gene, a protein-encoding nucleotide sequence with at least one CUA selector codon, and Methanococcus jannaschii tRNATyrCUA (Mj tRNATyrCUA). Using an in vivo complementation assay, cells expressing the Methanococcus jannaschii tRNATyrCUA (Mj tRNATyrCUA) alone survive to an IC₅₀ of 55 μg/mL ampicillin. Cells coexpressing Mj tRNATyrCUA with its TyrRS from M. jannaschii survive to an IC₅₀ of 1220 μg/mL ampicillin. Although Mj tRNATyrCUA is less orthogonal in E. coli than the SctRNAGlnCUA (IC₅₀ 20 μg/mL), the MjTyrRS has higher aminoacylation activity toward its cognate amber suppressor tRNA [see, e.g., L. Wang, T. J. Magliery, D. R. Liu and P. G. Schultz, J. Am. Chem. Soc., 122:5010 (2000)]. As a result, Methanococcus jannaschii/TyrRS is identified as an orthogonal pair in E. coli and can be selected for use in an in vivo translation system.

### Example 3

Synthesis of L. lactis Amber Suppressor Tyr tRNA Gene

Using the wildtype L. lactis Tyr tRNA sequence (SEQ ID NO. 7) as a starting point, a tRNA gene with modifications (SEQ ID NO. 8) was synthesized. These modifications included mutating the anticodon loop from GTA to CUA (anti-codon for amber, TAG, stop codon) and A16 to U (to introduce the sequence required for synthesis by RNA polymerase III) [see, Sakamoto, K, et al., Site-specific incorporation of an unnatural amino acid into proteins in mammalian cells, Nucleic Acids Res, 30:4692-9 (2002)]. For expression in mammalian cells, we also inserted the human Tyr 5′ leader sequence and 3′ RNA poly III termination sequence immediately upstream and downstream, respectively, of the L. lactis tyrosyl tRNA gene (SEQ ID NO. 9). We have also designed L. lactis Tyr tRNA genes for expression in yeast (SEQ ID NO. 10) and Xenopus oocytes (SEQ ID NO. 11).

### Example 4

Synthesis of L. lactis TyrRS Gene Based on Prior Art

The development of an orthogonal TyrRS/tRNA pair from E. coli that specifically recognizes the unnatural amino acid 4-methoxy-phenylalanine (4-MeO-Phe) using directed evolution has been described [Wang, L, Brock, A, Herberich, B, and Schultz, PG, Expanding the genetic code of Escherichia coli, Science, 292:498-500 (2001)]. Given that the amino acids critical for Tyr binding are highly conserved among TyrRS, as a starting point we generated the L. lactis TyrRS mutant containing the amino acid mutations described for the 4-MeO-Phe E. coli TyrRS (Tyr34Val, Aspl76Ser, and Phe177Met). Using the wildtype L. lactis TyrRS DNA sequence (DNA: SEQ ID NO. 1, Protein: SEQ ID NO. 2) as a starting point, a TyrRS gene was synthesized in which point mutations were incorporated to generate the foregoing amino acid mutations, the codon usage was “humanized,” and a hexahistidine tail was added to the C-terminus (DNA: SEQ ID NO. 3, Protein: SEQ ID NO. 4). The “humanized” L. lactis 4-MeO-Phe TyrRS gene was subcloned into a vector suitable for expression in mammalian cells. This L. lactis 4-MeO-Phe TyrRS gene however was not functional. This indicates that one cannot simply incorporate evolved E. coli TyrRS mutations for a given unnatural amino acid into L. lactis TyrRS.

### Example 5

Amber Suppression in Mammalian Cells

We demonstrated the orthogonality and functionality of an O-RS/O-tRNA pair derived from L. lactis in a mammalian cell by rescuing an amber TAG mutation in the hERG potassium channel. In this experiment, human embryonic kidney (HEK) cells were transfected with cDNAs encoding the genes for hERG 652TAG, L. lactis “humanized” wildtype TyrRS (DNA: SEQ ID NO. 5, Protein: SEQ ID NO. 6) and modified, L. lactis Tyr amber suppressor tRNA_{CUA} (SEQ ID NO. 7). Protein expression was assessed by Western Analysis using an antibody specific for hERG. The results are summarized in Table 2 below.

TABLE 2
Suppression of hERG 652TAG Mutation

| | Lane 1 | Lane 2 | Lane 3 | Lane 4 | Lane 5 | Lane 6 | Lane 7 | Lane 8 |
|---|---|---|---|---|---|---|---|---|
| hERG WT | − | + | − | − | − | − | − | − |
| hERG | − | − | + | + | − | − | + | + |
| 652TAG | | | | | | | | |
| RS | − | − | − | − | + | − | + | + |
| tRNA_{CUA} | − | − | − | + | − | + | + | + |

As a positive control, HEK cells were transfected with wildtype hERG (Table 2, lane 2). HEK cells transfected with hERG 652TAG cDNA expressed hERG only when both the L. lactis RS and suppressor tRNA_{CUA} cDNAs were also transfected into the cells (Table 2, lanes 7 and 8). This finding clearly demonstrates that 1) the cells are expressing L. lactis tyrosyl RS and suppressor tRNA_{CUA}, 2) the L. lactis tyrosyl RS aminoacylates its tyrosyl suppressor tRNA_{CUA} and 3) the L. lactis tyrosyl suppressor tRNA_{CUA} aminoacylated with tyrosine can “rescue” the hERG 652TAG mutation.

Equally important is that no hERG expression was observed in cells transfected with hERG 652TAG and suppressor tRNA_{CUA} cDNAs (Table 2, lane 4). This indicates that the L. lactis suppressor tRNA_{CUA} is not aminoacylated by the endogenous human tyrosyl RS (i.e., this confirms orthogonality). The lack of hERG expression in cells transfected only with hERG 652TAG cDNA indicates that read-through by an endogenous tRNA is not occurring (Table 2, lane 3).

### Example 6

Evaluation of Expressed hERG Protein

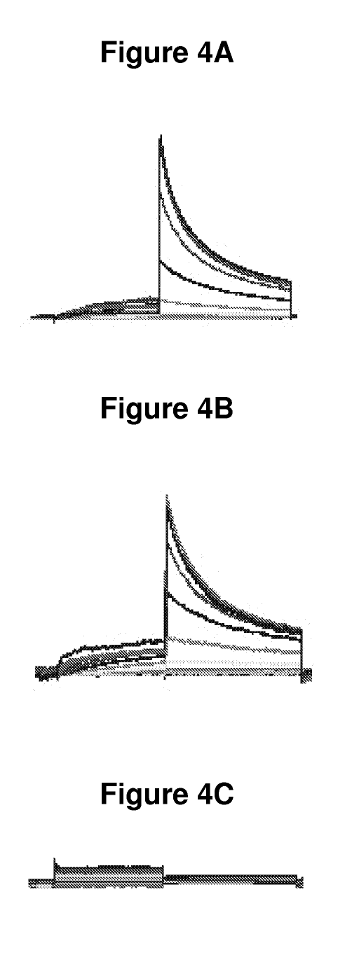

We repeated the transfection experiment described in Example 5 above in Chinese Hamster Ovary (CHO) cells and measured functional hERG currents using whole-cell electrophysiological recording techniques. Since electrophysiological measurements are at least 1000-fold more sensitive than Western Analysis for detecting protein expression, these measurements will provide a more stringent test for the orthogonality of the L. lactis TyrRS/suppressor tRNA_{CUA} pair. Shown in FIGS. 4A-4C are representative current traces recorded from CHO cells transfected with hERG wildtype plasmid (FIG. 4A), hERG652TAG+RS+suppressor tRNA_{CUA} plasmids (FIG. 4B) and hERG 6526+tRNA_{CUA} plasmids (FIG. 4C). The electrophysiological data shown in FIGS. 4A, 4B and 4C correspond to the Western data summarized in Table 2, lanes 2, 8 and 4, respectively).

Characteristic wildtype hERG current traces were obtained in cells transfected with wildtype hERG plasmid (FIG. 4A). As was observed for the Western data, CHO cells transfected with hERG 652TAG cDNA expressed wildtype hERG currents only when both the L. lactis RS and suppressor tRNA_{CUA} cDNAs were also transfected into the cells (FIG. 4B). Importantly, no wildtype hERG current was observed in cells transfected with hERG 652TAG and suppressor tRNA_{CUA} cDNAs (FIG. 4C) indicating that the suppressor tRNACUA was not acylated by endogenous RS molecules. Cells transfected with only hERG652TAG cDNA tended to have high leak currents and, therefore, electrophysiological currents could not be adequately measured. These data confirm the Western data and, given the sensitivity of electrophysiological measurements, these data clearly demonstrate the orthogonality of the L. lactis TyrRS/suppressor tRNA_{CUA} pair.

### Example 7

Cellular Uptake of Unnatural Amino Acids

Before generating a mutant L. lactis TyrRS for a given unnatural amino acid mutant it is important to verify that mammalian cells can uptake the unnatural amino acid and that the unnatural amino acid does not affect cell viability. Using LC-MS analysis, we have demonstrated that the unnatural amino acids 4-MeO-Phe, 3,5-F₂-Phe and cyclohexyl alanine (CHA) are readily taken up by HEK cells at levels comparable to that for Phe and Tyr.

We first determined the retention times of Tyr, Phe, and CHA, and then measured the ability of HEK cells to take up CHA. Following treatment of the cells with 1 mM of an unnatural amino acid for 48 hrs, cells were washed with PBS, lysed using 20% toluene and filtered through a 3 kDa cut-off filter. The filtrates were then analyzed by LC-MS. The mass spectrum for the peak corresponding to CHA confirms that this peak is indeed due to CHA. In untreated cells, we readily detected Tyr and Phe but there was no peak corresponding to CHA. These data confirm that HEK cells take up CHA and that CHA concentrations comparable to those observed for Tyr and Phe are obtained. Similar findings were observed for 3,5-F₂-Phe and 4-OMe-Tyr. These experiments indicate that we can readily obtain the intracellular concentrations of unnatural amino acids required for aminoacylation of the L. lactis tRNA_{CUA}.

### Example 8

A Mutant L. lactis TyrRS Library for Directed Evolution

To evolve a L. lactis O-RS specific for an unnatural amino acid, variants comprising all possible natural mutations are generated at key residues shown to be involved in amino acid binding. For a TyrRs, these key residues are: Tyr37, Asn123, Asp176, Phe177 and Leu180 [see, Brick, P, Bhat, T N, and Blow, D M, Structure of tyrosyl-tRNA synthetase refined at 2.3 A resolution, Interaction of the enzyme with the tyrosyl adenylate intermediate, J Mol Biol, 208:83-98 (1989)]. Further, directed evolution using a library of random mutations at these five positions can then be utilized in isolating unnatural amino acid mutants [see, e.g., Chin, J W, Cropp, T A, Chu, S, Meggers, E, and Schultz, P G, Progress toward an expanded eukaryotic genetic code, Chem Biol, 10:511-519 (2003); Santoro, S W, Wang, L, Herberich, B, King, D S, and Schultz, P G, An efficient system for the evolution of aminoacyl-tRNA synthetase specificity, Nat Biotechnol, 20:1044-1048 (2002)].

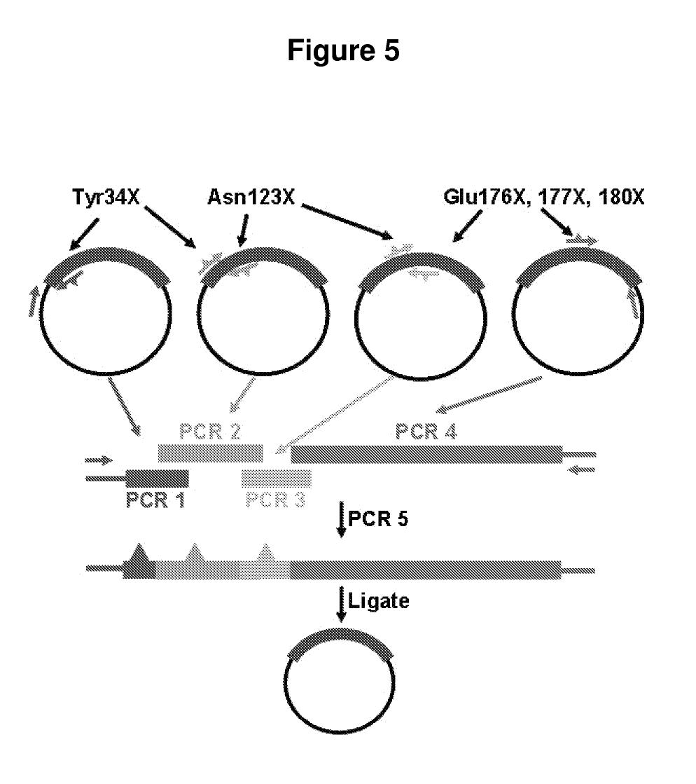

To generate a library encoding all possible mutations at positions 37, 123, 176, 177 and 180, the strategy outlined in FIG. 5 is employed. This strategy makes use of overlapping PCR primers and oligonucleotides that contain degenerate codons corresponding to these positions [see, Parikh, M R and Matsumura, I, Site-saturation mutagenesis is more efficient than DNA shuffling for the directed evolution of beta-fucosidase from beta-galactosidase, J Mol Biol, 352:621-628 (2005)]. In this way a final PCR product coding for full-length TyrRS that contains 3.2×10⁶ individual mutants is generated. The final PCR product(s) are then subcloned into the BamHI/NotI sites of ptRNA_{CUA}/ADH1-TyrRS (described below) to yield a mutant library (ptRNA_{CUA}/ADH1-mutRS).

### Example 9

Yeast Selection System for Isolating Mutant L. lactis TyrRS Molecules

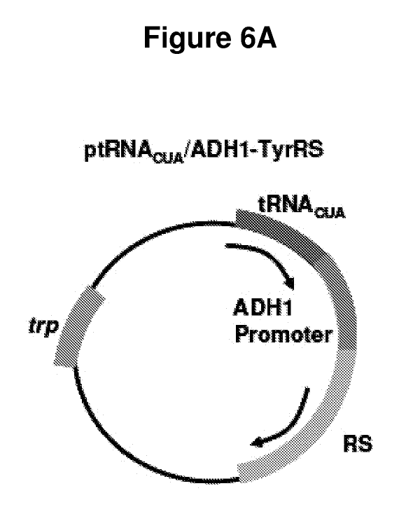

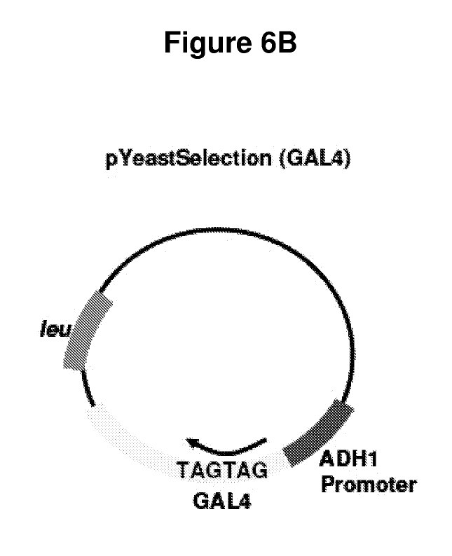

A yeast-two hybrid screening system is used for isolating L. lactis TyrRS mutants that specifically recognize unnatural amino acids. This approach was used to select E. coli TyrRS mutants [see, Chin, et al., Progress toward an expanded eukaryotic genetic code, Chem Biol 10:511-9 (2003)]. Two plasmids are used transfected into the yeast cells in this system in order to isolate mutant L. lactis TyrRS. One is a plasmid selected from a plasmid library containing suppressor tRNA_{CUA} and TyrRS mutants (ptRNA/ADH1-TyrRS, shown in FIG. 6A) and the other is a plasmid containing the GAL4 gene that has two TAG mutations (pYeastSelection, shown in FIG. 6B).

To generate ptRNA/ADH1-TyrRS, the L. lactis suppressor tRNA_{CUA} construct (SEQ ID NO. 9) designed for expression in mammalian cells, comprising 5′ and 3′ UTR regions of the human Tyr tRNA gene, was modified, as it has been reported that human tRNA genes do not generally express well in yeast unless the 5′ and 3′ UTRs are replaced. Since it has been shown that the E. coli Tyr tRNA gene expresses in yeast [see Chin, et al., Progress toward an expanded eukaryotic genetic code, Chem Biol 10:511-9 (2003)], we generated by site-directed mutagenesis (QuickChange, Stratagene) a L. lactis suppressor tRNA_{CUA} construct containing the 5′ and 3′ UTRs from E. coli Tyr tRNA gene (SEQ ID NO. 10).

The L. lactis TyrRS and tRNA_{CUA} genes were subcloned into the yeast expression vector pESC-TRP (Stratagene). To drive the expression of TyrRS, we inserted the yeast ADH1 promoter immediately upstream of the TyrRS gene. We generated restriction enzyme sites on the TyrRS and tRNA_{CUA} genes by PCR. The PCR products and pESC-TRP were digested with the appropriate restriction enzymes, and the fragments were ligated to generate ptRNA_{CUA}/ADH1-TyrRS (FIG. 6A). Yeast transformed with ptRNA_{CUA}/ADH1-TyrRS can be selected by growing on media lacking tryptophan

To select for L. lactis synthetases specific for unnatural amino acids, we also generated a plasmid containing GAL4 that has two TAG mutations at positions 44 and 110. These two amino acid positions are permissive with respect to incorporation of a large variety of amino acids [see, Chin, et al., Progress toward an expanded eukaryotic genetic code, Chem Biol 10:511-9 (2003)], though they are not the only two positions that can be utilized. To generate pYeastSelect we isolated the yeast GAL4 gene from pCL1by digestion with HindlIl. The GAL4 HindlIl fragment was then subcloned into the HindIII site of pGADGH. The TAG mutations at positions 44 and 110 were generated by site-directed mutagenesis (QuickChange). Yeast transformed with pYeastSelection can be selected by growing on media lacking leucine.

The yeast strain MaV203 is then transformed with each of the plasmids described above and grown in the presence of an unnatural amino acid in order to select for RS molecules which charge tRNAs with the unnatural amino acid. MaV203 has been engineered such that the transcription factor encoded by the GAL4 gene product has been knocked out and the genes encoding proteins required for the biosynthesis of uracil (URA3) and histidine (HIS3) are under control of the GAL4 promoter. Yeast expressing a functional GAL4 transcription factor will grow on media lacking uracil or histidine. MaV203 yeast are Functional mutant RS molecules aminoacylate the suppressor tRNA_{CUA}, resulting in rescue of the GAL4TAG mutant and expression of functional GAL4. GAL4 then drives the synthesis of the URA3 and HIS3 gene products.

Positive selection is then performed by growing yeast on media which lacks uracil, or histidine-lacking media that contains 1 mM 3-AT histidine, and which contains the unnatural amino acid. This selects for RS molecules that use natural amino acids, unnatural amino acids, or both. Only those yeast that express a functional mutRS (using either a natural or the unnatural amino acid) will survive.

Negative selection is then performed by growing the surviving yeast on media containing 5-fluoroorotic acid (5-FOA) in the absence of the unnatural amino acid. The URA3 gene product converts 5-FOA to the toxic 5-fluorouracil, which causes yeast death and thereby selects for RS molecules that use only the unnatural amino acid. The surviving yeast are those that express a functional mutRS synthetase that uses the unnatural amino acid. After two to three rounds of positive/negative selection the plasmids containing the mutant RS are isolated from the surviving yeast.

### Example 10

Directed Evolution to Develop RS Mutants

Directed evolution is used to isolate an “evolved” L. lactis TyrRS that recognizes a specific unnatural amino acid. The steps involved in developing unnatural amino acid mutant TyrRS molecules are: 1) verification of cellular uptake of the unnatural amino acid, 2) generation of a mutant TyrRS library and 3) selection of a mutant TyrRS specific for the unnatural amino acid using a yeast expression system.

Although the present invention has been discussed in considerable detail with reference to certain preferred embodiments, other embodiments are possible. The steps disclosed for the present methods are not intended to be limiting nor are they intended to indicate that each step depicted is essential to the method, but instead are exemplary steps only. As will be understood by those of skill in the art with reference to this disclosure, the actual dimensions of any device or part of a device disclosed herein, and the actual volumes, amounts, time periods, and other quantities recited in the process and method steps in this disclosure, will be determined by the intended use of such device or the intended application of such process or method. Therefore, the scope of the appended claims should not be limited to the description of preferred embodiments contained in this disclosure. All references cited herein are incorporated by reference to their entirety.

What is claimed is:

1. A translation system comprising:
(a) translation components derived from a eukaryotic organism; and
(b) an aminoacyl synthetase/tRNA pair derived from an organism selected from the group consisting of Lactococcus lactis, Gluconobacter oxydans and Rhodospirullum rubrum, wherein the aminoacyl synthetase and tRNA are orthogonal with respect to the translation components derived from the eukaryotic organism, and wherein the tRNA is aminoacylated with an unnatural amino acid by the aminoacyl synthetase with enhanced efficiency as compared to aminoacylation of the tRNA by the aminoacyl synthetase with a natural amino acid.

2. The translation system of claim 1, wherein the tRNA comprises an anticodon loop having a sequence that specifically binds to a selector codon.

3. The translation system of claim 2, wherein the selector codon is selected from the group consisting of an amber codon, an opal codon, an ocher codon, and a four base codon.

4. The translation system of claim 1, wherein the aminoacyl synthetase/tRNA pair is derived from Lactococcus lactis.

5. The translation system of claim 1, wherein the aminoacyl synthetase is derived from a tyrosyl aminoacyl synthetase.

6. The translation system of claim 1, wherein the translation components comprises endogenous translation components in a cell, and wherein the aminoacyl synthetase/tRNA pair is present in the cell.

7. The translation system of claim 6, wherein the cell is selected from the group consisting of a yeast cell, an insect cell, and a mammalian cell.

8. The translation system of claim 6, further comprising a nucleic acid molecule comprising one or more sequences encoding the aminoacyl synthetase/tRNA pair.

9. The translation system of claim 6, wherein the nucleic acid molecule comprises a sequence selected from the group consisting of any of SEQ ID NOS. 1-11.

10. The translation system of claim 1, further comprising the unnatural amino acid.

11. The translation system of claim 10, wherein the unnatural amino acid is selected from the group consisting of a tyrosine analog, a glutamine analog, a phenylalanine analog, serine analog, a threonine analog, a β-amino acid, and a cyclic amino acid other than proline.

12. The translation system of claim 10, wherein the unnatural amino acid is a derivative of a natural amino acid comprising a substitution or addition selected from the group consisting of an alkyl group, an aryl group, an acyl group, an azido group, a cyano group, a halo group, a hydrazine group, a hydrazide group, a hydroxyl group, an alkenyl group, an alkynl group, an ether group, a thiol group, a sulfonyl group, a seleno group, an ester group, a thioacid group, a borate group, a boronate group, a phospho group, a phosphono group, a phosphine group, a heterocyclic group, an enone group, an imine group, an aldehyde group, a hydroxylamino group, a keto group, a sugar group, α-hydroxy group, a cyclopropyl group, a cyclobutyl group, a cyclopentyl group, a 2-nitrobenzyl group, a 3,5-dimethoxy-2-nitrobenzyl group, a 3,5-dimethoxy-2-nitroveratrole carbamate group, a nitrobenzyl group, a 3,5-dimethoxy-2-nitrobenzyl group, and an amino group.

13. The translation system of claim 10, wherein the unnatural amino acid is a derivative of a natural amino acid comprising an addition selected from the group consisting of a photoactivatable cross-linker, a spin-label, a fluorescent label, a radioactive label, biotin, a biotin analog, and a photocleavable group.

14. The translation system of claim 10, wherein the unnatural amino acid is selected from the group consisting of hydroxy methionine, norvaline, O-methylserine. crotylglycine, hydroxy leucine, allo-isoleucine, norleucine, α-aminobutyric acid, t-butylalanine, hydroxy glycine, hydroxy serine, F-alanine, hydroxy tyrosine, homotyrosine, 2-F-tyrosine, 3-F-tyrosine, 4-methyl-phenylalanine, 4-methoxy-phenylalanine, 3-hydroxy-phenylalanine, 4-NH₂-phenylalanine, 3-methoxy-phenylalanine, 2-F-phenylalanine, 3-F-phenylalanine, 4-F-phenylalanine, 2-Br-phenylalanine, 3-Br-phenylalanine, 4-Br-phenylalanine, 2-Cl-phenylalanine, 3-Cl-phenylalanine, 4-Cl-phenylalanine, 4-CN-phenylalanine, 2,3-F₂-phenylalanine, 2,4-F₂-phenylalanine, 2,5-F₂-phenylalanine, 2,6-F₂-phenylalanine, 3,4-F₂-phenylalanine, 3,5-F₂-phenylalanine, 2,3-Br₂-phenylalanine, 2,4-Br₂-phenylalanine, 2,5-Br₂-phenylalanine, 2,6-Br₂-phenylalanine, 3,4-Br₂-phenylalanine, 3,5-Br₂-phenylalanine, 2,3-Cl₂-phenylalanine, 2,4-Cl₂-phenylalanine, 2,5-Cl₂-phenylalanine, 2,6-Cl₂-phenylalanine, 3,4-Cl₂-phenylalanine, 2,3,4-F₃-phenylalanine, 2,3,5-F₃-phenylalanine, 2,3,6-F₃-phenylalanine, 2,4,6-F₃-phenylalanine, 3,4,5-F₃-phenylalanine, 2,3,4-Br₃-phenylalanine, 2,3,5-Br₃-phenylalanine, 2,3,6-Br₃-phenylalanine, 2,4,6-Br₃-phenylalanine, 3,4,5-Br₃-phenylalanine, 2,3,4-Cl₃-phenylalanine, 2,3,5-Cl₃-phenylalanine, 2,3,6-Cl₃-phenylalanine, 2,4,6-Cl₃-phenylalanine, 3,4,5-Cl₃-phenylalanine, 2,3,4,5-F₄-phenylalanine, 2,3,4,5-Br₄-phenylalanine, 2,3,4,5-Cl₄-phenylalanine, 2,3,4,5,6-F₅-phenylalanine, 2,3,4,5,6-Br₅-phenylalanine, 2,3,4,5,6-Cl₅-phenylalanine, cyclohexylalanine, hexahydrotyrosine, cyclohexanol-alanine, hydroxyl alanine, hydroxy phenylalanine, hydroxy valine, hydroxy isoleucine hydroxyl glutamine, thienylalanine, pyrrole alanine, N_{T}-methyl-histidine, 2-amino-5-oxohexanoic acid, norvaline, norleucine, 3,5-F₂-phenyalanine, cyclohexyalanine, 4-Cl-phenyalanine, p-azido-phenylalanine, o-azido-phenylalanine, O-4-allyl-L-tyrosine, 2-amino-4-pentanoic acid, and 2-amino-5-oxohexanoic acid.

15. A method of incorporating an unnatural amino acid into a protein in a eukaryotic cell, comprising the steps of:
(a) providing the cell of claim 6;
(b) providing the unnatural amino acid; and
(c) producing the protein, wherein the unnatural amino acid is incorporated into the protein.

16. The method of claim 15, further comprising the steps of:
(a) providing a first nucleic acid molecule comprising a sequence that codes for the protein, wherein the sequence comprises a selector codon;
(b) providing a second nucleic acid molecule that encodes the aminoacyl synthetase derived from Lactococcus lactis which is orthogonal with respect to the translation system;
(c) providing a third nucleic acid molecule that encodes a tRNA derived from Lactococcus lactis which is orthogonal with respect to the translation system; and
(d) transfecting the cell with the first, second, and third nucleic acid molecules.

17. A vector comprising:
(a) a first nucleic acid molecule comprising a first nucleic acid sequence that encodes an aminoacyl synthetase derived from Lactococcus lactis; and
(b) a second nucleic acid molecule comprising a second nucleic acid sequence that encodes a tRNA derived from Lactococcus lactis that is aminoacylated with an unnatural amino acid by the aminoacyl synthetase derived from Lactococcus lactis, wherein the tRNA comprises an anticodon loop having a sequence that specifically binds a selector codon of an mRNA molecule.

18. The vector of claim 17, wherein the vector comprises a first plasmid comprising the first nucleic acid sequence and a second plasmid comprising the second nucleic acid sequence.
